(19) United States
(12) Patent Application Publication
(10) Pub. No.: US 2005/0112763 A1
(43) Pub. Date: May 26, 2005

(54) RNAI-based modification of heterochromatin

(75) Inventors: Shivinder S. Grewal, Gaithersburg, MD (US); Thomas Volpe, Evanston, IL (US); Ira M. Hall, Melville, NY (US); Catherine Kidner, East Lothian (GB); Robert A. Martienssen, Cold Spring Harbor, NY (US)

Correspondence Address: DARBY & DARBY P.C., P. O. BOX 5257, NEW YORK, NY, 10150-5257, US

(73) Assignee: Cold Spring Harbor Laboratory

(21) Appl. No.: 10/923,268

(22) Filed: Aug. 20, 2004

Related U.S. Application Data
(60) Provisional application No. 60/497,218, filed on Aug. 21, 2003.

(51) Int. Cl. A61K 48/00

(52) U.S. Cl. 435/372; 514/44

(57) ABSTRACT

Methods of regulating the formation of heterochromatin at a target locus within the genome of a cell and methods of regulating the modification of a histone protein assumed on the DNA of a target locus within the genome of a cell are provided. In particular, the invention provides methods to regulate the structure of the genome of a cell, where the structure is regulated by modulating heterochromatin formation, heterochromatin architecture, histone methylation, and/or DNA methylation, by exposing the cell to an RNAi probe complementary to a target locus so as to activate the RNAi machinery to modify the genomic structure of the target locus and adjacent segments of the genome.

This application claims priority under 35 U.S.C. §119(e) to U.S. Ser. No. 60/497,218, filed on Aug. 21, 2003, which is incorporated herein by reference in its entirety.

The research leading to this invention was supported, in part, by Grant No. GM59772 awarded by the National Institutes of Health (NIH). Accordingly, the United States government may have certain rights to this invention.

### FIELD OF THE INVENTION

The invention pertains to the field of RNA interference and genome structure. In particular, the invention provides methods to regulate the structure of the genome of a cell, where the structure is regulated by modulating heterochromatin formation, heterochromatin architecture, histone methylation, and/or DNA methylation, by exposing the cell to an RNAi probe complementary to a target locus so as to activate the RNAi machinery to modify the genomic structure of the target locus and adjacent segments of the genome.

### BACKGROUND OF THE INVENTION

In eukaryotes, the organization of chromatin into higher-order structures governs diverse chromosomal processes. Besides creating distinct metastable transcriptional domains during cellular differentiation, the formation of higher-order chromatin domains known as heterochromatin, or silent chromatin, is widely recognized to be essential for imprinting, dosage compensation, recombination, and chromosome condensation (Reik and Walter. Curr Opin Genet Dev 1998;8:154; Grewal and Elgin. Curr Opin Genet Dev 2002;12:178; Cohen and Lee. Curr Opin Genet Dev 2002;12:219; and Cavalli. Curr Opin Cell Biol 2002;14:269).

Eukaryotic chromosomes are characterized by the presence of condensed tracts of constitutive heterochromatin which stain differentially and have a low density of expressed genes, but have a high density of transposons and repeats. The assembly of heterochromatin at centromeres is essential for the accurate segregation of chromosomes during cell division, and the formation of such specialized structures at telomeres protects chromosomes from degradation and from aberrant chromosomal fusions (Grewal and Elgin. Curr Opin Genet Dev 2002;12:178). The juxtaposition of heterochromatic regions with euchromatic genes results in gene silencing, often in a variegated fashion in which founder cells pass on alternate epigenetic states to their descendants (Henikoff. Curr Opin Genet Dev 1992;2:907; Wallrath. Curr Opin Genet Dev 1998;8:147; and Grewal and Elgin. Curr Opin Genet Dev 2002;12:178).

Repetitive DNA sequences such as transposable elements are often assembled into heterochromatin that, in addition to its role in transcriptional repression, maintains genome integrity by suppressing recombination between repetitive elements (Henikoff. Biochim Biophys Acta 2000;1470:1). Transposable elements can also regulate neighboring genes by conferring epigenetic expression states, and it has been proposed that many of the properties of heterochromatin stem from these elements (Martienssen and Colot. Science 2001;293:1070).

The posttranslational modification of histone tails plays a causal role in the assembly of higher order chromatin, and accumulating evidence suggests that patterns of histone modification specify discrete downstream regulatory events (Strahl and Allis. Nature 2000;403:41 and Jenuwein and Allis. Science 2001;293:1074). For example, in S. pombe, heterochromatin is marked by methylation of histone H3 at lysine 9 (K9 or Lys9), while methylation of lysine 4 (K4) is preferentially associated with expressed genes in active chromatin (Nakayama et al. Science 2001;292:110 and Noma et al. Science 2001;293:1150). Similarly, in mammals, methylation of histone H3 at the lysine 9 and/or lysine 27 residue is associated with inactive heterochromatin (see, e.g., Nicolas et al. Mol Cell Biol 2003;23:1614-1622; Silva et al. Dev Cell 2003; 4:481-495; and Plath et al. Science 2003;300:131-135).

The factors that define particular chromosomal domains as preferred sites of heterochromatin assembly are largely uncharacterized. It has been suggested that heterochromatin protein complexes are preferentially targeted to repetitive DNA elements, such as commonly found at the pericentric heterochromatin and intergenic regions of higher eukaryotes (Birchler et al. Curr Opin Genet Dev 200;10:211 and Hsieh and Fire. Annu Rev Genet 2000;34: 187). Interestingly, rather than any specific sequence motif, the repetitive nature of transgene arrays alone appears to be sufficient for heterochromatin formation (Hsieh and Fire. Annu Rev Genet 2000;34:187 and Dorer and Henikoff. Cell 1994;77:993). Furthermore, studies of position effect variegation have shown that heterochromatin complexes possess the ability to spread along the chromosomes, resulting in the heritable inactivation of nearby sequences (Grewal and Elgin. Curr Opin Genet Dev 2002;12:178).

Higher-order chromatin structure is critical for the functional organization of centromeres and the mating-type region of the fission yeast Schizosaccharomyces pombe (Grewal and Elgin. Curr Opin Genet Dev 2002;12:178). At centromeres, tandem and inverted arrays of the dg and dh centromeric repeats surrounding the unique central core are assembled into heterochromatin, and are bound by CENP-B proteins that resemble the transposase encoded by POGO-like elements (Chikashige et al. Cell 1989;57:739; Hahnenberger et al. Mol Cell Biol 1991;11:2206; Allshire et al. Genes Dev 1995;9:218; and Nakagawa et al. Genes Dev 2002;16:1766). At the mating-type region, a 20-kb domain containing the mat2 and mat3 silent donor loci and the interval between them, known as the K-region, are subject to heterochromatin-mediated silencing and recombination suppression (Grewal and Elgin. Curr Opin Genet Dev 2002;12:178).

Heterochromatin formation at the centromeres and within the silent mating-type (mat2/3) interval requires many of the same trans-acting factors, including histone deacetylases (HDACs), the H3 Lys9-specific methyltransferase Clr4, and Swi6, the fission yeast counterpart to mammalian HP1 (Ekwall and Ruusala. Genetics 1994;136:53; Thon et al. Genetics 1994;138:29; Grewal et al. Genetics 1998;150:563; and Nakayama et al. Science 2001;292:110). Genetic studies have shown that clr4 is responsible for histone H3 lysine 9 methylation and acts upstream of swi6 (Nakayama et al. Science 2001; 292: 110). Both genes encode a protein containing a chromodomain. Chromodomains have been implicated in binding RNA as well as histone H3 methylated on lysine 9 (Akhtar at al. Nature 2000:407;405). These previous findings have suggested a model for heterochromatin formation in which the cooperative activity of HDACs and the H3 Lys9 methyltransferase Clr4 establish a “histone code” that is essential for the localization of Swi6 to silenced genomic locations (Nakayama et al. Science 2001;292:1110).

Formation of heterochromatin within the entire 20-kb of the silent mating-type region of yeast is dependent upon H3 Lys9 methylation by Clr4 and the subsequent binding of Swi6, both of which are restricted to this domain by the IR-R and IR-L boundary elements (Noma et al. Science 2001; 293:1150). The K-region, in particular the 4.3-kb cenH sequence that contains several clusters of short direct repeats and shares strong homology with dg and dh centromeric elements, is important for heterochromatin assembly (Grewal and Klar. Genetics 1997; 146:1221). Replacement of the cenH-containing region with ura4⁺ (KΔ::ura4⁺) results in a metastable locus that displays alternative silenced (ura4-off) and expressed (ura4-on) epigenetic states (Nakayama et al. Cell 2000;1010:307 and Grewal and Klar. Cell 1996;86:95). This variegation of ura4⁺ expression is due to defects in the establishment of the silenced chromatin state in KΔ::ura4⁺ cells. Once assembled, the ura4-off state is remarkably stable during both mitosis and meiosis. Moreover, maintenance of the ura4-off state requires functional Swi6, Clr4, and histone deacetylases (HDACs).

These findings add to the expanding list of cellular functions that depend on the proper regulation of higher-order chromatin structure. Structural regulation is not just critical for the formation and maintenance of chromatin transcriptional domains; it is also essential to proper chromatin dynamics, which are central to cell division and sexual reproduction. Heterochromatin assembly at the centromeres facilitates both kinetochore formation and sister chromatid cohesion. (Allshire et al. Genes Dev. 1995; 9;218; Murphy and Karpen. Cell 1995;82:599; Kellum and Alberts. J. Cell Sci. 1995;108:1419). In addition, the formation of specialized chromatin structures at telomeres serves to maintain the length of telomeric repeats, to suppress recombination, and to aid in formation of a bouquet-like structure that facilitates homologous chromosome pairing during meiosis. (Allshire et al. Genes Dev. 1995;9:218; Grewal et al. Genetics 1998;150:563).

The growing recognition of higher-order chromatin structure in a wide array of cellular functions has prompted the need for specific approaches to manipulate distinct chromatin domains. This invention provides such an approach by revealing an unexpected link between RNA interference (RNAi) and chromatin regulation.

RNAi is the process by which double stranded RNA (dsRNA) inhibits the accumulation of homologous transcripts from cognate genes (Montgomery et al. Proc Natl Acad Sci USA 1998;95:15502). It is thought to be responsible for post-transcriptional gene silencing (PTGS), or co-suppression, a mechanism by which endogenous genes are silenced in the presence of a homologous transgene (Jorgensen. Trends Biotechnol 1990;8:340). Several of the genes required for RNAi have been isolated from Arabidopsis, C. elegans and Drosophila. These include an RNaseIII helicase, an RNA dependent RNA polymerase, and several members of the ARGONAUTE gene family. The Drosophila RNaseIII helicase Dicer has been shown to specifically cleave dsRNA into sense and antisense RNA oligonucleotides 21-24 nts in length (Bernstein et al. Nature 2001; 409:363). These small interfering RNAs (siRNAs) were first observed in plants (Hamilton and Baulcombe. Science 1999;286:950) and are believed to guide the RNA interference silencing complex (RISC) to messenger RNA transcribed from homologous genes (Hammond et al. Nature 2000;404:293 and Tuschl et al. Genes Dev 1999; 13:3191). One component of RISC is Argonaute 2 (Hammond et al. Science 2001;293:1146), a gene highly conserved in animals and plants (Fagard et al. Proc Natl Acad Sci USA 2000;97:11650). The dsRNA is thought to be re-generated, or amplified, by an RNA-dependent RNA polymerase (RdRP) that uses siRNA to prime dsRNA synthesis (Lipardi et al. Cell 2001;107:297 and Sijen et al. Cell 2001;107:465). Although the primary sequence of RdRP is not related to viral replicases, RdRP mutants can be complemented by single stranded RNA (ssRNA) viruses in plants (Dalmay et al. Cell 2000;101:543) which are important targets of RNAi (Vaistil et al. Plant Cell 2002;14:857).

Many of the genes required for RNAi are redundant in plants and animals, although some RNAi mutants can be lethal or sterile, suggesting that endogenous genes are direct or indirect targets. For example, in Arabidopsis, the related genes ARGONAUTE and PINHEAD/ZWILLE have synergistic effects on stem cell maintenance and organogenesis and double mutants result in embryo lethality (Lynn et al. Development 1999;126:469).

The fission yeast S. pombe contains homologs of the Argonaute (ago1), Dicer (dcr1), and RNA-dependent RNA polymerase (rdp1) genes required for RNAi-related processes in other systems (Hannon. Nature 2002;418:244; Aravind et al. Proc Natl Acad Sci USA 2000;97:11319; and Hutvagner and Zamore. Curr Opin Genet Dev 2002;12:25). However, unlike higher eukaryotes, S. pombe has only a single homolog of the argonaute gene (Wood et al. Nature 2002;415:871). S. pombe therefore provides an attractive system to study the cellular functions of RNAi.

Through experiments in S. pombe, the instant invention discloses unexpected links between RNAi machinery and chromatin structure. In turn, these findings provide new and specific approaches, based on RNAi, to manipulate the structure and function of the cellular genome.

### SUMMARY OF THE INVENTION

The present invention is directed to a method of regulating the formation of heterochromatin at a target locus within the genome of a cell, which method comprises introducing to the cell an RNAi probe that is complementary to a portion of the target locus, wherein the RNAi probe induces the assembly of heterochromatin at the region of the target locus that is complementary to the RNAi probe. In certain embodiments, the assembly of heterochromatin at the region of the target locus that is complementary to the RNAi probe produces heritable, pre-transcriptional silencing of a gene spanning the target locus, or of a gene adjacent to the target locus.

The present invention is further directed to a method of regulating the modification of a histone protein assembled on the DNA of a target locus within the genome of a cell, which method comprises introducing to the cell an RNAi probe that is complementary to a portion of the target locus, wherein the RNAi probe induces the methylation of the histone protein assembled on the DNA at the region of the target locus that is complementary to the RNAi probe. In certain embodiments, the methylation of the histone protein assembled on the DNA at the region of the target locus that is complementary to the RNAi probe produces heritable, pre-transcriptional silencing of a gene spanning the target locus, or of a gene adjacent to the target locus.

The present invention is also directed to a method of regulating the modification of the DNA at a target locus within the genome of a cell, which method comprises introducing to the cell an RNAi probe that is complementary to a portion of the target locus, wherein the RNAi probe induces the methylation of the DNA of a target locus at the region of the target locus that is complementary to the RNAi probe. In certain embodiments, the methylation of the DNA of a target locus at the region of the target locus that is complementary to the RNAi probe produces heritable, pre-transcriptional silencing of a gene spanning the target locus, or of a gene adjacent to the target locus.

These methods may be used in any of several therapeutic contexts, including in neoplasia and cancer; retroviral infection; regulation of transposons and repeat elements; genomic imprinting and X-inactivation; aneuploidy, partial aneuploidy, and gene duplication; disorders of chromatin assembly and genome instability; and experimental models of deletion syndromes.

### DESCRIPTION OF THE DRAWINGS

FIG. 1 depicts data showing that centromeric silencing is relieved in ago1⁻, dcr1⁻, and rdp1⁻ mutant strains compared to wildtype. A. Diagram of the first S. pombe centromere, cen1. The left and right outermost (otrL and otrR, respectively), the left and right innermost (imrL and imrR, respectively) and central (cnt1) centromeric regions are indicated. Within the ortL and otrR regions, the conserved dg (white arrows) and dh (grey arrows) are indicated. The three different locations of ura4⁺ transgene integration into cen1 (cnt, imr, and otr) are indicated (vertical black arrows). B. Northern analysis of RNA transcripts transcribed from centromeric ura4⁺ transgenes and a ura4⁺-DS/E mini-gene located on the chromosome arm. Wildtype (WT), dcr1⁻, rdp1⁻, and ago1⁻ S. pombe strains carrying either the otr, imr or cnt ura4⁺ centromeric transgene as well as the ura4-DS/E chromosome arm minigene were assayed for ura4⁺ transgene expression as well as ura4⁺-DS/E mini-gene by Northern blot. C. Transcripts derived from the centromeric repeats were detected by Northern blotting. Wildtype (wt), dcr1⁻, rdp1⁻, and ago1⁻ S. pombe strains were assayed for expression of dg centromeric repeat derived transcripts (cen) by Northern blot using probes specific for dg centromeric repeats. Detection of act1 transcripts served as a positive control. D. Transcripts derived from the centromeric repeats were detected by Northern blotting. Wildtype (wt), ago1⁻, swi6⁻, and ago1⁻ swi6 S. pombe strains were assayed for expression of dg centromeric repeat derived transcripts (cen) by Northern blot using probes specific for dg centromeric repeats. Detection of act1 transcripts served as a positive control.

FIG. 2 depicts data showing that expression of the centromeric otr transcripts is both transcriptionally and post-transcriptionally regulated by the RNAi machinery. A. A schematic diagram showing the direction of transcription of forward and reverse transcripts corresponding to the centromeric otr region dh repeat. B. Strand specific RT-PCR analysis was performed in the presence (+RT) of reverse transcriptase. RNA samples prepared from wildtype (wt), dcr1, rdp1⁻, ago1⁻, swi6⁻, and ago1⁻ swi6 S. pombe strains were incubated with primers from the dh repeat complementary to either the forward (cen For) or reverse (cen Rev) centromeric transcripts in first strand cDNA synthesis reaction. Both primers were present in subsequent cycles of PCR amplification after heat inactivation of the reverse transcriptase. Positive control strand specific RT-PCR reactions were also conducted using primers specific for act1 sense (act1 s) or act1 antisense (act1 as) transcripts. C. Strand specific RT-PCR analysis was performed in the absence (-RT) of reverse transcriptase. RNA samples prepared from wildtype (wt), dcr1⁻, rdp1⁻, ago1⁻, swi6⁻, and ago1⁻ swi6 S. pombe strains were incubated with primers from the dh repeat complementary to either the forward (cen For) or reverse (cen Rev) centromeric transcripts in first strand cDNA synthesis reaction. Both primers were present in subsequent cycles of PCR amplification after heat inactivation of the reverse transcriptase. Positive control strand specific RT-PCR reactions were also conducted using primers specific for act1 sense (act1 s) or act1 antisense (act1 as) transcripts. D. Strand specific analysis of nascent RNA transcripts was performed by nuclear run-on assay. Radiolabeled nascent RNA purified from wildtype (wt), dcr1⁻, rdp1⁻, ago1⁻, swi6⁻, and ago1⁻ swi6 S. pombe strains was hybridized to nylon membranes containing the strand specific probes for the forward (cen For) or reverse (cen Rev) centromeric transcripts, and for the sense (act1 s) or antisense (act1 as) actin transcripts.

FIG. 3 depicts data from chromatin immunoprecipitation experiments showing that chromatin structure at centromeric repeats is altered in ago1⁻, dcr1, and rdp1 RNAi machinery mutants. A. Quantitative real-time PCR was performed on DNA fragments immunoprecipitated with antibodies raised against H3 Lys⁴-methyl (Meth H3 Lysine 4) or H3 Lys 9-methyl, or branched H3 peptide tails methylated on Lys⁹ (Meth H3 Lysine 9 Branched) using primers specific for centromeric dg repeats (cen). These experimental results were quantitated relative to standards of real-time PCR using primers specific for the actin gene (Meth H3 Lysine 4) or for the S. pombe the matK mating type repeat (Meth H3 Lysine 9 and Meth H3 Lysine 9 Branched). The DNA from S. pombe wildtype (wt), dcr1⁻, rdp1⁻, and ago1⁻ strains was assayed. B. DNA fragments purified from whole cell extracts (wce) or DNA fragments immunoprecipitated with antibodies raised against Swi6 protein (Swi6) or Lys⁹-methyl (K9) histone H3, were amplified by PCR using ura4 specific primers, that amplify both the ura4⁺ transgene located in the otr of cen1 (otr::ura4⁺), as well as the ura4 DS/E minigene (ura4 DS/E) located on the chromosome arm. Relative levels were estimated using a FUJI phosphoimager and are indicated below each lane.

FIG. 4 depicts a model based upon the discovery that the RNAi machinery is required for the initiation and maintenance of the heterochromatic state of centromeric repeats. Reverse strand centromeric transcription occurs in wildtype cells and is degraded post-transcriptionally by the RNAi machinery. Low level transcription from the forward strand and/or amplification by Rdp1 results in generation of dsRNA, which is converted to siRNA by the RNAi machinery. Rdp1, bound to the chromatin, allows targeting of histone modifications to specific sequences via siRNA, resulting in maintenance of the heterochromatic state. “otr” is a centromeric otr region repeat. “Rdp1” is the RNA dependent polymerase protein. “HMT” is a histone methyl-transferase protein that methylates the histones of the otr repeat. The black lollipops indicate Lys9-methyl histone proteins. “Swi6” is Swi6 protein that binds to Lys9-methylated histone.

FIG. 5 depicts data showing that Swi6 is required for H3 Lys9 methylation at the mat region. A. Schematic representations of the mating-type regions of the S. pombe strains Kint2::ura4⁺ and KΔ::ura4⁺. In Kint2::ura4⁺, the ura4⁺ transgene is inserted at the cenH region, while in KΔ::ura4⁺ the cenH-containing region is replaced with the ura4⁺ transgene. These strains also contains the a ura4-DS/E mini-gene inserted into the chromosome arm of chromosome 3. B. Effects of mutation in swi6 (swi6-115) on Swi6 and H3 Lys9-methylation levels at the ura4⁺ transgene in S. pombe strain Kint2::ura4⁺. ChIP analysis was performed on S. pombe strain Kint2::ura4⁺, where the strain was wildtype for swi6 (swi6⁺) or contained the swi6-115 mutation (swi6)., DNA fragments purified from whole cell extracts (WCE) or DNA fragments immunoprecipitated with antibodies raised against Lys⁹-methyl (K9) histone H3 or Swi6 protein (Swi6), were amplified by PCR using ura4 specific primers, that amplify both the ura4⁺ transgene inserted at the cenH region of (Kint2::ura4⁺), as well as the ura4 DS/E minigene (ura4 DS/E) located on the chromosome arm. The fold enrichment (fold enrichment of Kint2::ura4⁺), calculated relative to the WCE control (−), is shown underneath each lane. C. Effects of mutation in swi6 (swi6-115) on Swi6 and H3 Lys9-methylation levels at the ura4⁺ transgene in S. pombe strain KΔ::ura4⁺. ChIP analysis was performed on S. pombe strain KΔ::ura4⁺, where the strain was wildtype for swi6 (swi6⁺) or contained the swi6-115 mutation (swi6⁻). DNA fragments purified from whole cell extracts (WCE) or DNA fragments immunoprecipitated with antibodies raised against Lys⁹-methyl (K9) histone H3 or Swi6 protein (Swi6), were amplified by PCR using ura4 specific primers, that amplify both the ura4⁺ transgene replacing the cenH region (KΔ::ura4⁺), as well as the ura4 DS/E minigene (ura4 DS/E) located on the chromosome arm. The fold enrichment (fold enrichment of KΔ::ura4⁺), calculated relative to the WCE control (−), is shown underneath each lane. D. Swi6 protein and H3 Lys9-methylation are differentially localized in the ura4-off and ura4-on epialleles of KΔ::ura4⁺. ChIP analysis was performed on S. pombe strain KΔ::ura4⁺ cells that had silenced the ura4⁺ transgene (ura4-off) and that had failed to silence the ura4⁺ transgene (ura4-on). DNA fragments purified from whole cell extracts (WCE) or DNA fragments immunoprecipitated with antibodies raised against Lys⁹-methyl (K9) histone H3 or Swi6 protein (Swi6), were amplified by PCR using ura4 specific primers, that amplify both the ura4⁺ transgene replacing the cenH region (KΔ::ura4⁺), as well as the ura4 DS/E minigene (ura4 DS/E) located on the chromosome arm. The fold enrichment (fold enrichment of KΔ::ura4⁺), calculated relative to the WCE control (−), is shown underneath each lane.

FIG. 6 depicts data showing that differential H3 Lys9 methylation and Swi6 localization patterns shown by ura4-off and ura4-on epialleles of KΔ::ura4⁺ are stably inherited in cis. A diagram of the cross is shown at the top: KΔ::ura4⁺ strains carrying ura4-off his2⁻ and ura4-on his2⁺ epialleles were crossed, allowed at least 30 generation of diploid growth, sporulated, and subjected to tetrad analysis. Resulting colonies were replicated onto non-selective medium (N/S), medium lacking uracil (AA-URA), histidine (AA-HIS), or containing 5-fluoroorotic acid (FOA). Segregants from individual tetrads were subjected to ChIP analysis. DNA fragments purified from whole cell extracts (WCE) or DNA fragments immunoprecipitated with antibodies raised against Lys⁹-methyl histone H3 (H3 Lys9-methyl) or Swi6 protein (Swi6), were amplified by PCR using ura4 specific primers, that amplify both the ura4⁺ transgene replacing the cenH region (KΔ::ura4⁺), as well as the ura4 DS/E minigene (ura4 DS/E) located on the chromosome arm. The fold enrichment (fold enrichment of ura4⁺), calculated relative to the WCE control, is shown underneath each lane.

FIG. 7 depicts data showing the effects of mutation in swi6 on H3 Lys9 and H3 Lys4 methylation at the mat2/3 interval of the S. pombe mating-type region. A physical map of the silent mating-type region is shown at the top. Specific positions whose histone methylation status was measured are indicated by numbers below the physical map. ChIP analysis was performed on wildtype (WT) and swi6 mutant (swi6−) S. pombe strains. DNA fragments purified from whole cell extracts (WCE) or DNA fragments immunoprecipitated with antibodies raised against H3 Lys⁹-methyl histone H3 (H3 Lys9-methyl) or H3 Lys4-methyl histone H3 (H3 Lys4-methyl), were amplified by PCR using primers specific for a DNA fragment of the mat locus (mat), and primers specific for the actin (act1) gene. The ratios of the mat locus and control act1 signals present in whole cell extract (WCE) DNA were used to calculate relative fold enrichment of immunoprecipitated samples. Quantitation of these results is plotted in alignment with a map of the mat locus, where light grey is the results for WT cells, and dark grey is the results for swi6⁻ cells.

FIG. 8 depicts the S. pombe mating scheme whereby a mating type region (mat) derived from either a clr4⁺ or a clr4Δ background was introduced into the RNAi mutant backgrounds by genetic crosses. Diploids were constructed by crossing the indicated strains, sporulated, and subjected to tetrad analysis.

FIG. 9 depicts data derived from immunofluorescent studies of Swi6, showing that the number of Swi6 foci is increased in diploids homozygous for deletions (Δ or ⁻) of ago1, dcr1, and rdp1, respectively. The graph shows the frequency of Swi6 foci number. More than 150 cells were counted for each strain.

### DETAILED DESCRIPTION OF THE INVENTION

The present invention relates to the discovery that the RNAi machinery is required for the initiation of heterochromatin formation, that the RNAi machinery is required for appropriate methylation of histones in areas of gene silencing and heterochromatin assembly, and that RNAi machinery-mediated heterochromatin assembly and histone methylation at a target locus produces heritable, pre-transcriptional gene silencing. These discoveries also provide a link between RNA interference and DNA methylation, indicating that the genome modifications promoted by the RNAi machinery in turn promote the methylation of DNA.

The present invention also relates to the discovery that the RNAi machinery is required for the fidelity of chromosome segregation, that centromeric cohesion is defective in RNAi mutant strains, that RNAi mutants are defective in telomere clustering, that meiotic chromosome segregation is severely perturbed in RNAi mutants, and that RNAi mutants show defects in meiotic telomere clustering.

Based upon these novel discoveries, novel methods to manipulate the structure and function of the genome are disclosed herein. Specifically, the present invention provides methods of regulating heterochromatic formation at a target locus, of regulating the methylation of histone proteins at a target locus, and of regulating the methylation of DNA at a target locus.

These methods will be useful in several therapeutic contexts, including in neoplasia and cancer; retroviral infection; regulation of transposons and repeat elements; genomic imprinting and X-inactivation; aneuploidy, partial aneuploidy, and gene duplication; and disorders of chromatin assembly and genome instability.

As used herein, the term “histone” refers to any of a group of highly conserved proteins, designated H1, H2A, H2B, H3, and H4, that associate with and organize eukaryotic DNA. The histones and DNA combine to form nucleosomes, which consist of a core of histone proteins around which is coiled approximately 140 base pairs of DNA. These nucleosomes are then further packaged into stacks to form “chromatin”, which is comprised of DNA and histones as well as other proteins.

As used herein, the term “heterochromatin” refers to sections of chromatin assembled into a condensed form. Heterochromatin is commonly visible in cytogenetic analysis as differentially staining chromosomal domains of highly compacted higher-order structures. The posttranslational modification of histone tails plays a causal role in the assembly of heterochromatin, and accumulating evidence suggests that specific patterns of histone modification are associated with heterochromatin (Strahl and Allis. Nature 2000;403:41 and Jenuwein and Allis. Science 2001;293:1074). For example, in S. pombe, heterochromatin is marked by methylation of histone H3 at lysine 9 (K9), while methylation of lysine 4 (K4) is preferentially associated with expressed genes in active chromatin (Nakayama et al. Science 2001;292:110 and Noma et al. Science 2001;293:1150). DNA assembled into heterochromatin is generally transcriptionally inactive. Heterochromatin, in addition to its role in transcriptional repression, suppresses recombination between repetitive elements. Heterochromatin complexes possess the ability to spread along the chromosomes, resulting in the heritable inactivation of nearby sequences (Grewal and Elgin. Curr Opin Genet Dev 2002;12:178).

As used herein, the term “genome” refers to the entire nuclear DNA content of a cell. This nuclear DNA includes both the endogenous DNA content of the cell as well as any exogenous DNA sequences (e.g., as from retroviruses, transposable elements, or introduced transgenes) which have been heritably attached to the endogenous DNA sequences (i.e., stably integrated in to the endogenous sequences). In mammals, for example, the genome is subdivided into distinct portions of DNA contained within the individual chromosomes of the nucleus.

As used herein, the term “target locus” refers to any segment of the genome of a cell whose formation into heterochromatin, whose histone modifications, whose DNA methylation and/or whose heritable pre-transcriptional gene silencing status is to be manipulated according to the methods of the invention. Such target loci include, for example, transposable elements, repeat sequence elements (e.g., Alu elements), endogenous genes, transcribed and untranscribed segments of integrated retroviruses or other integrated exogenous sequences (e.g., as from transgenes), and untranscribed intergenic sequences. In the methods of the invention, the introduced RNAi probe is complementary to the nucleic acid sequence of a portion of the target locus.

As used herein, the term “gene” refers to a segment of the genome comprising a nucleotide sequence that is transcribed into RNA. Genes include, for example; endogenous DNA encoding mRNAs, tRNAs and rRNAs; segments of integrated transgenes and retroviruses; and transposons. A gene is comprised of both transcribed and untranscribed portions. Transcribed portions of a gene include introns, exons, 5′ untranslated sequences, and 3′ untranslated sequences. Non-transcribed portions of a gene include the promoter and enhancer sequences. A gene is referred to as “spanning the target locus” if the target locus falls within the gene. A gene is referred to as “adjacent to the target locus” if the target locus does not fall within the gene, but is located upstream or downstream of the gene.

As used herein, the term “promoter” refers to a DNA regulatory region capable of binding RNA polymerase in a cell and initiating transcription of a downstream (3′ direction) coding sequence. For purposes of defining the present invention, the promoter sequence is bounded at its 3′ end by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

As used herein, the term “RNA interference probe” or “RNAi probe” refers to synthetic or natural ribonucleic acid species, or derivatives thereof, which activate the RNAi machinery, e.g. to induce RNA interference (RNAi)-mediated assembly of heterochromatin and/or heritable pre-transcriptional gene silencing, when introduced into a cell. “RNAi probes” include small interfering RNAs (siRNAs) and short hairpin RNAs (shRNAs). These RNAi probes comprise sequences that are complementary to, and therefore specific for, a segment of the sequence of the target locus. The term “RNAi probe” also encompasses the expression constructs used for in vivo synthesis of siRNAs and shRNAs.

In particular the methods of the invention comprise introducing to a cell an RNAi probe that is complementary to the nucleic acid sequence of a portion of the target locus. In certain embodiments, the portion of the target locus to which the RNAi probe is complementary is at least about 15 nucleotides in length. In preferred embodiments, the portion of the target locus to which the RNAi probe is complementary is at least about 19 nucleotides in length.

The cells to be used in accordance with the present invention include yeast cells and animal cells, but not plant cells or prokaryotic cells. Suitable animal cells include, for example, insect cells (such as Drosophila cells), nematode cells (such as C. elegans cells), and mammalian cells, including human cells. In the case of animal cells, the cells may be cultured in vitro or present in vivo in an animal. Note that the method must be performed on a cell that is capable of affecting the type of genome modification encompassed by the method. For example, S. cerevisiae yeast cells have no RNAi interference machinery, and therefore should not be used in the methods of the invention. As another example, S. pombe yeast cells possess the RNAi machinery and show methylation of histones and assembly of heterochromatin, but have no DNA methylation. Thus, the methods directed to manipulating DNA methylation should not be performed on S. pombe cells. Conversely, mammalian cells possess the RNAi machinery, and show assembly of heterochromatin, methylation of histones, and DNA methylation. Therefore, mammalian cells are preferred cells of the invention. Particularly preferred mammalian cells include human cells.

### RNA Interference (RNAi)

RNA interference (RNAi) is a process of sequence-specific post-transcriptional gene silencing by which double-stranded RNA (dsRNA) homologous to a target locus can specifically inactivate gene function in plants, fungi (such as S. pombi), invertebrates, and mammalian systems (Hammond et al. Nat Genet 2001;2:110; Sharp. Genes Dev 1999;13:139). This dsRNA-induced gene silencing is mediated by 21- and 22-nucleotide double-stranded small interfering RNAs (siRNAs) generated from longer dsRNAs by ribonuclease III cleavage (Bernstein et al. Nature 2001;409:363; and Elbashir et al. Genes Dev 2001;15:188). RNAi-mediated gene silencing is thought to occur via sequence-specific mRNA degradation, where sequence specificity is determined by the interaction of an siRNA with its complementary sequence within a target mRNA (See, e.g., Tuschl. Chem Biochem 2001;2:239).

For mammalian systems, RNAi may be activated by introduction of either siRNAs (Elbashir et al. Nature 2001;411:494) or short hairpin RNAs (shRNAs) bearing a fold back stem-loop structure (Paddison et al. Genes Dev 2002;16:948; Sui et al. Proc Natl Acad Sci USA 2002;99:5515; Brummelkamp et al. Science 2002;296:550; and Paul et al. Nat Biotechnol 2002;20:505).

### Small Interfering RNAs (siRNAs)

The siRNAs to be used in the methods of the present invention are short double stranded nucleic acid duplexes comprising annealed complementary single stranded nucleic acid molecules. In preferred embodiments, the siRNAs are short double stranded RNAs comprising annealed complementary single strand RNAs. However, the invention also encompasses embodiments in which the siRNAs comprise an annealed RNA:DNA duplex, wherein the sense strand of the duplex is a DNA molecule and the antisense strand of the duplex is a RNA molecule.

Preferably, each single stranded nucleic acid molecule of the siRNA duplex is of from about 21 nucleotides to about 27 nucleotides in length. In preferred embodiments, duplexed siRNAs have a 2 or 3 nucleotide 3′ overhang on each strand of the duplex. In preferred embodiments, siRNAs have 5′-phosphate and 3′-hydroxyl groups.

According to the present invention, siRNAs may be introduced to a target cell as an annealed duplex siRNA, or as single stranded sense and anti-sense nucleic acid sequences that once within the target cell anneal to form the siRNA duplex. Alternatively, the sense and anti-sense strands of the siRNA may be encoded on an expression construct that is introduced to the target cell. Upon expression within the target cell, the transcribed sense and antisense strands may anneal to reconstitute the siRNA.

### Short Hairpin RNAs (shRNAs)

The shRNAs to be to be used in the methods of the present invention comprise a single stranded “loop” region connecting complementary inverted repeat sequences that anneal to form a double stranded “stem” region. Structural considerations for shRNA design are discussed, for example, in McManus et al. RNA 2002;8:842-850. In certain embodiments the shRNA may be a portion of a larger RNA molecule, e.g., as part of a larger RNA that also contains U6 RNA sequences (Paul et al. Nature Biotech 2002;20:505-508).

In preferred embodiments the loop of the shRNA is from about 0 to about 9 nucleotides in length. In preferred embodiments the double stranded stem of the shRNA is from about 19 to about 33 base pairs in length. In preferred embodiments, the 3′ end of the shRNA stem has a 3′ overhang. In particularly preferred embodiments, the 3′ overhang of the shRNA stem is from 1 to about 4 nucleotides in length. In preferred embodiments, shRNAs have 5′-phosphate and 3′-hydroxyl groups.

### Chemical Synthesis of RNAi Probes

RNA molecules may be chemically synthesized, for example using appropriately protected ribonucleoside phosphoramidites and a conventional DNA/RNA synthesizer. Suppliers of RNA synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). For example, single-stranded gene-specific RNA oligomers may be synthesized using 2′-O-(tri-isopropyl) silyloxymethyl chemistry by Xeragon AG (Zurich, Switzerland). Alternatively, RNA oligomers may be synthesized using Expedite RNA phosphoramidites and thymidine phosphoramidite (Proligo). RNAs produced by such methodologies tend to be highly pure and to anneal efficiently to form siRNA duplexes or shRNA hairpin stem-loop structures.

Following chemical synthesis, single stranded RNA molecules are deprotected, annealed to form siRNAs or shRNAs, and purified (e.g., by gel electrophoresis or High Pressure Liquid Chromatography). For example, siRNAs may be generated by annealing sense and antisense single strand RNA (ssRNA) oligomers. Similarly, shRNAs may be generated by annealing of complementary sequences within a single ssRNA molecule to form a hairpin stem-loop structure. The integrity and the dsRNA character of the annealed RNAs may be confirmed by gel electrophoresis and quantified by spectroscopy (using the standard conversion, wherein 1 unit of Optical Density at 260 nm=40 μg of duplex RNA/ml).

Most conveniently, siRNAs may be obtained from commercial RNA oligomer synthesis suppliers, which sell RNA-synthesis products of different quality and cost. For example, commercial suppliers of siRNAs include Dharmacon, Xeragon Inc. (now a QIAGEN company), Proligo, and Ambion.

### In Vitro Enzymatic Synthesis of RNAi Probes

Standard procedures may used for in vitro transcription of RNA from DNA templates carrying RNA polymerase promoter sequences (e.g., T7 or SP6 RNA polymerase promoter sequences). Efficient in vitro protocols for preparation of siRNAs using T7 RNA polymerase have been described (Donzé and Picard. Nucleic Acids Res 2002;30:e46; and Yu et al. Proc Natl Acad Sci USA 2002;99:6047). Similarly, an efficient in vitro protocol for preparation of shRNAs using T7 RNA polymerase has been described (Yu et al. Proc Natl Acad Sci USA 2002;99:6047).

For example, sense and antisense RNA oligonucleotides for siRNA preparation may be transcribed from a single DNA template that contains a T7 promoter in the sense direction and an SP6 promoter in the antisense direction. Alternatively, sense and antisense RNAs may be transcribed from two different DNA templates containing a single T7 or SP6 promoter sequence. The sense and antisense transcripts may be synthesized in two independent reactions or simultaneously in a single reaction. Similarly, a ssRNA may be synthesized from a DNA template encoding a shRNA. The transcribed ssRNA oligomers are then annealed and purified. siRNAs may be generated by annealing sense and antisense ssRNA oligomers. Similarly, shRNAs may be generated by annealing of complementary sequences within a single ssRNA molecule to form a hairpin stem-loop structure. The integrity and the dsRNA character of the annealed RNAs may be confirmed by gel electrophoresis and quantified by spectroscopy (using the standard conversion, wherein 1 unit of Optical Density at 260 nm=40 μg of duplex RNA/ml).

### In Vivo Synthesis of RNAi Probes within Cells

RNAi probes may be formed within a cell by transcription of RNA from an expression construct introduced into the cell. For example, a protocol and expression construct for in vivo expression of siRNAs is described in Yu et al. supra. Similarly, protocols and expression constructs for in vivo expression of shRNAs have been described (Brummelkamp et al. Science 2002;296:550; Sui et al. Proc Natl Acad Sci USA 2002;99:5515; Yu et al. supra; McManus et al. RNA 2002;8:842; and Paul et al. Nature Biotech 2002;20:505).

For example, an siRNA may be reconstituted in a cell by use of an siRNA expression construct that upon transcription within the cell produces the sense and antisense strands of the siRNA. These complementary sense and antisense RNAs then anneal to reconstitute the siRNA within the cell. In one embodiment, the sense and antisense strands are encoded by a single sequence of the expression vector flanked by two promoters of opposite transcriptional orientation, thereby driving transcription of the alternate strands of the sequence. In another embodiment, the sense and antisense strands are encoded by independent sequences within a single expression vector, where each independent sequence is operably linked to a promoter to drive transcription. In yet another embodiment, the sense and antisense strands are encoded by independent sequences on two independent expression constructs, where each independent sequence is operably linked to a promoter to drive transcription.

Similarly, shRNAs may be generated in vivo by transcription of a single stranded RNA from an expression construct within a cell. The complementary sequences of the inverted repeat within the ssRNA then anneal to yield the stem-loop structure of the shRNA.

Expression construct-encoded RNAi probes have distinct advantages over their chemically synthesized or in vitro transcribed counterparts. They are cost effective and provide a stable and continuous expression of RNAi probe that is useful for analysis of phenotypes that develop over extended periods of time.

The expression constructs for in vivo production of RNAi probes comprise RNAi probe encoding sequences operably linked to elements necessary for the proper transcription of the RNAi probe encoding sequence(s), including promoter elements and transcription termination signals. Preferred promoters for use in such expression constructs include the polymerase-III HI-RNA promoter (see, e.g., Brummelkamp et al. supra) and the U6 polymerase-III promoter (see, e.g., Sui et al. supra; Paul et al. supra; and Yu et al. supra).

The RNAi probe expression constructs may further comprise vector sequences that facilitate the cloning and propagation of the expression constructs. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest. Prolonged expression of the encoded RNAi probe in in vitro cell culture may be achieved by the use of vectors sequences that allow for autonomous replication of an extrachromosomal construct in mammalian host cells (e.g., EBNA-1 and oriP from the Epstein-Barr virus).

### Sequence Composition of RNAi Probes

The RNAi probes to be used in the methods of the present invention comprise nucleic acid sequences that are complementary to the nucleic acid sequence of a portion of the target locus. In certain embodiments, the portion of the target locus to which the RNAi probe is complementary is at least about 15 nucleotides in length. In preferred embodiments, the portion of the target locus to which the RNAi probe is complementary is at least about 19 nucleotides in length. The target locus to which an RNAi probe is complementary may represent a transcribed portion of the genome (i.e., a gene) or an untranscribed portion of the genome (e.g., intergenic regions, repeat elements, etc.).

The RNAi candidates to be screened according to the invention preferably contain nucleotide sequences that are fully complementary to a portion of the target locus. However, 100% sequence complementarity between the RNAi probe and the target locus is not required to practice the invention. Therefore, RNA sequences with insertions, deletions, and single point mutations relative to the sequence of the target locus may be used in the methods of the present invention.

The degree of sequence complementarity between an RNAi probe and its target locus may be determined by sequence comparison and alignment algorithms known in the art (see, for example, Gribskov and Devereux Sequence Analysis Primer (Stockton Press: 1991) and references cited therein). The percent similarity between the nucleotide sequences may be determined, for example, using the Smith-Waterman algorithm as implemented in the BESTFIT software program using default parameters. Greater than 90% sequence complementarity between the RNAi probe and the portion of the target locus corresponding to the RNAi probe is preferred.

### Modifications to RNAi Probes

The RNA of RNAi probes may include one or more modifications, either to the phosphate-sugar backbone or to the nucleoside. For example, the phosphodiester linkages of natural RNA may be modified to include at least one heteroatom, such as nitrogen or sulfur. In this case, for example, the phosphodiester linkage may be replaced by a phosphothioester linkage. Similarly, bases may be modified to block the activity of adenosine deaminase. Where the RNAi candidate or probe is produced synthetically, or by in vitro transcription, a modified ribonucleoside may be introduced during synthesis or transcription. For example, incorporation of 2′-aminouridine, 2′-deoxythymidine, or 5′-iodouridine into the sense strand of an RNAi probe is tolerated by the RNAi pathway, whereas the same substitutions on the antisense strand of the RNAi is not (Parrish et al. Mol Cell 2000;6:1077). Also, if a siRNA has a 2 or 3 nucleotide 3′ overhang on each strand of the duplex, substitution of 2′-deoxythymidine for uridine in the overhangs is tolerated by the RNAi pathway.

### Methods of the Present Invention

Based on the discoveries that the RNAi machinery is required for the initiation of heterochromatin formation, that the RNAi machinery is required for appropriate methylation of histones in areas of gene silencing and heterochromatin assembly, that RNAi machinery-mediated heterochromatin assembly and histone methylation at a target locus produces heritable pre-transcriptional gene silencing, and that there is a link between RNAi and DNA methylation, novel methods to manipulate the structure and function of the genome are provided herewith.

In particular, the invention provides methods for regulating the formation of heterochromatin at a target locus, wherein the introduction of an RNAi probe complementary to a portion of a target locus into a cells activates the RNAi machinery to assemble heterochromatin at the target locus. Once formed on the portion of the target locus complementary to the RNAi probe, the heterochromatin may spread to adjacent sequences of the target locus, such that a domain of the target locus substantially larger than that encompassed by the RNAi probe is assemble into heterochromatin. In addition, the assembled heterochromatin may spread further into adjacent regions of the genome. Thus, the methods of the present invention provide the ability to induce the assembly of heterochromatin across various regions of the genome.

In particular, the RNAi machinery exerts its effects on heterochromatin formation, at least in part, by directing the methylation of histone proteins at specific amino acid residues of the histone. For example, in yeast, methylation of histone H3 on the lysine 9 residue is associated with inactive heterochromatin (Nakayama et al. Science 2001;292:1110 and Noma et al. Science 2001;293:1150). Similarly, in mammals, methylation of histone H3 at the lysine 9 and/or lysine 27 residue is associated with inactive heterochromatin (see, e.g., Nicolas et al. Mol Cell Biol 2003;23:1614; Silva et al. Dev Cell 2003;4:481; and Plath et al. Science 2003;300:131). The presence of methyl groups on these specific amino acid residues stimulates the further assembly of the modified histones into heterochromatin. Thus, the results reported herein for the effects of RNAi on heterochromatin and histone methylation in S. pombe cells constitute useful analogs by which to understand the effects of RNAi on heterochromatin and histone methylation in mammalian cells.

According to the methods of the invention, the RNAi machinery directs histone methylation of histone located on specific nucleic acid sequences, where this sequence specificity is determined by the RNAi probe associated with the RNAi machinery. This region of RNAi-specified histone methylation may then spread outward from the initial target sequence so as to direct modification of histones across a broader segment of the genome.

Thus, the present invention also provides methods to regulate the modification of histones, wherein the introduction of an RNAi probe complementary to a portion of a target locus into a cell activates the RNAi machinery to direct the methylation of the histone proteins assembled on the DNA at the target locus.

The present invention further provides methods to regulate the methylation of DNA at a target locus wherein the introduction of an RNAi probe complementary to a portion of a target locus into a cell activates the RNAi machinery and thereby ultimately leads to the methylation of DNA at the target locus. Methylation of DNA on the cytosine residue of a CpG dinucleotide as frequently associated with gene silencing (e.g., as has been observed for various imprinted genes such as IgfR2).

These effects of an RNAi probe on heterochromatin formation, histone modification, and/or DNA methylation at a target locus, and loci adjacent to the target locus, have dramatic effects on the function of these genomic segments. For example, DNA that is assembled into heterochromatin is generally transcriptionally inactive. In addition, assembly into heterochromatin suppresses recombination.

The assembly of heterochromatin and/or modification of histones in a cell exposed to an RNAi probe complementary to a target locus may be assessed by any of several techniques well established in the art, including cytogenetic analysis, observation of gene expression of genes spanning or adjacent to the target locus, and observation of the methylation and/or acetylation status of histones assembled on a target locus. Similarly, the methylation status of target locus DNA in a cell exposed to an RNAi probe complementary to a target locus may be assessed by any of several techniques well established in the art, observation of gene expression of genes spanning or adjacent to the target locus, and observation of the methylation status of DNA of a target locus.

Techniques for observing gene expression are well established in the art and include, for example, reverse transcription-polymerase chain reaction (RT-PCR), northern blot analysis, western blot analysis, enzyme linked immunosorbent assay (ELISA), in situ hybridization, etc.

Techniques for observing the methylation and/or acetylation status of histones assembled on a target locus are well established in the art and include immunoassays (e.g., immunoprecipitation) coupled to detection of specific sequences (e.g., by polymerase chain reaction (PCR), hybridization analysis (e.g., southern blots and “slot blots”).

Antibodies specific for histones containing methyl or acetyl groups on defined amino acid residues are commercially available (e.g., from Cell Signaling Technology, CHEMICON, or Novus Biologicals).

Similarly, techniques for observing the methylation status of target locus DNA are well established in the and include immunoassays (e.g., immunoprecipitation) coupled to detection of specific sequences (e.g., by polymerase chain reaction (PCR), hybridization analysis (e.g., southern blots and “slot blots”).

Antibodies specific for DNA containing methyl groups on cytosine residues are commercially available (e.g., from AbCam or Novus Biologicals).

These and other related methods are fully explained in the art (see, e.g., Sambrook et al. Molecular Cloning: A Laboratory Manual, Third Edition (Cold Spring Harbor Laboratory Press: 2001); Ausubel et al., eds. Current Protocols in Molecular Biology (John Wiley & Sons, Inc.: 1994); Harlow and Lane. Using Antibodies: A Laboratory Manual (Cold Spring Harbor Laboratory Press: 1999; DNA Cloning: A Practical Approach, Volumes I and II (Glover, ed. 1985); Ausubel et al. (eds.); PCR Primer: A Laboratory Manual, Second Edition; Dieffenbach and Dveksler, eds. (Cold Spring Harbor Laboratory Press: 2003); and Hockfield et al. Selected Methods for Antibody and Nucleic Acid Probes (Cold Spring Harbor Laboratory Press: 1993).

### Applications of the Methods of the Present Invention

The ability to manipulate heterochromatin formation and histone modification, and thereby to regulate genome function, may be advantageously applied in several therapeutic contexts.

### Neoplasia and Cancer

As is well known in the art, neoplasia and cancer can arise from several different molecular causes, including expression of oncogenic proteins (e.g., Ras, and the BCR/ABL fusion protein of chronic myeloid leukemia), expression of mutant tumor suppressor proteins (e.g., p53 mutants), and activity of oncogenic retroviruses (e.g., Human T-cell leukemia virus (HTLV), Rous sarcoma virus (RSV), Feline Leukemia Virus (FeLV)). In addition, cancer cells frequently show marked genomic instability, including increased occurrence of translocations, inversions, and aneuploidy. The methods of the present invention provide robust methods to treat cancer as caused by any of these molecular mechanisms.

By directing the formation of heterochromatin, histone modifications, and/or DNA methylation at a target locus spanning or adjacent to a gene encoding an endogenous oncogene or endogenous mutant tumor suppressor gene, the methods of the invention can be used to provide heritable, pre-transcriptional gene silencing of these gene products.

In the case of retroviruses, some retroviruses are oncogenic due to expression of virally encoded oncogenes, while others are oncogenic due to their effects on expression of endogenous oncogenes at the site of viral insertion (see, e.g., Coffin et al. Retroviruses (Cold Spring Harbor Laboratory Press: 1997)). In particular, HTLV, a retrovirus that does not express a virally encoded oncogene, is associated with lymphoid malignancies in humans. In contrast, Rous sarcoma virus, associated with fibrosarcoma in chickens, is oncogenic due to the virally encoded Src protein.

The methods of the present invention may be used to direct the formation of heterochromatin, histone modifications, and/or DNA methylation across the sequences of an integrated oncogenic retrovirus. Where the retrovirus is oncogenic due to expression of a virally encoded oncogene, the formation of heterochromatin, histone modifications, and/or DNA methylation on the viral sequences will provide heritable, pre-transcriptional gene silencing of the virally encoded oncogene. Where the retrovirus is oncogenic due to its effects on the expression level of neighboring endogenous oncogenes, the formation of heterochromatin, histone modifications, and/or DNA methylation on the viral sequences will abrogate the affect of the viral sequences on the expression of the endogenous gene. Where the retrovirus is oncogenic due to its mutagenic effects on an endogenous gene, the formation of heterochromatin, histone modifications, and/or DNA methylation on the viral sequences will provide heritable, pre-transcriptional gene silencing of the mutated endogenous gene.

As noted above, cells of benign neoplasms and malignant cancers frequently show genomic instability. In particular, the degree of genomic instability generally increases during the progression from benign neoplasm to malignancy. The methods of the present invention provide the means to intervene in this process.

Genomic instability often results from an increased rate of recombination, particularly between repeat elements dispersed across the genome. The methods of the present invention provide the ability to direct the packaging of such repeat elements into heterochromatin, thereby suppressing recombination between the repeats and decreasing the occurrence of translocation and inversion events in the genome.

Similarly, the progression from benign neoplasm to malignancy is often associated with increased frequency of aneuploidy due to aberrant chromosome segregation at cell division. The methods of the present invention provide the ability to combat this aberrant chromosome segregation by ensuring the proper assembly of centromeric and telomeric sequences into heterochromatin. For example, where a benign or malignant growth shows decondensation of the centromeric repeats or telomeric repeats, the genome stability of these cells may be enhanced by an RNAi probe that directs the re-packaging of the centromeric repeats or telomeric repeats into heterochromatin.

According to the methods of the invention, an RNAi probe complementary to the appropriate target locus (e.g., centromeric repeats, integrated oncogenic retrovirus, endogenous gene, etc.) is introduced into the neoplastic or malignant cell in order to induce the assembly of heterochromatin at the target locus and/or direct histone modification at the target locus. The therapeutic outcome may then be assessed in any of several ways. For example, the treated cells may show a decreased rate of cell proliferation, a patient may show a reduction in tumor volume, a benign growth may fail to progress to malignancy, or the treated cells may show improved genomic stability upon cytogenetic analysis.

### Retroviruses

The methods of the present invention will also be useful in the treatment of conditions due to infection with a retrovirus, including Acquired Immune Deficiency Syndrome (AIDS) due to infection with the Human Immunodeficiency Virus (HIV), and those associated with neoplasms and cancer (see above).

In particular, the methods of the present invention provide a means to heritably silence the integrated viral genome, and thereby treat the viral infection.

For example, in the case of HIV, an RNAi probe complementary to a portion of the HIV genome may be introduced to an infected cell. The RNAi probe will then activate the RNAi machinery to assemble heterochromatin across, direct modification of histone on, and/or direct DNA methylation on the integrated viral genome. The viral sequences will thereby be heritably silenced.

The therapeutic outcome may then be assessed in any of several ways. For example, by analysis of viral load, by analysis of CD4⁺ T-cell counts, reduced frequency of opportunistic infection (e.g., pneumonia or thrush), or by amelioration of other presenting clinical symptoms (e.g., presence of Kaposi's sarcoma lesions, fever, fatigue, etc.).

### Transposons and Repeat Elements

The methods of the present invention also provide the means to regulate the effect of transposons and repeat elements on genome structure and function.

The activity of transposons can lead to genomic instability due to insertional mutagenesis of actively transposing transposons, and due to recombination between the sequences of active or inactive transposon sequences. Similarly, repeat elements can lead to genomic instability due to high levels of recombination between repeat elements.

High levels of recombination, as between transposons or repeat elements, contribute to genomic instability in several ways. For example, inversion and translocation events can be mutagenic and have deleterious consequences. In addition, translocation events can contribute to aberrant chromosome segregation, for example where a translocation results in an aberrant chromosome having two centromeres, or having no centromeres. Similarly, translocation events can lead to partial aneuploidy, e.g., wherein following translocation and cell division a cell has three copies of the short arm of a given chromosome (two in their endogenous location, and a third inappropriately attached to the centromere of a different chromosome).

The methods of the present invention provide the means to combat the genomic instability due to transposons and repeat elements by providing a method to direct assembly of these sequences into an inactive form. For example, an RNAi probe complementary to a given transposon or repeat sequence may be introduced to a cell. The RNAi probe will then activate the RNAi machinery to assemble heterochromatin across, direct modification of histone on, and/or stimulate DNA methylation of the transposon or repeat sequences in their various locations throughout the genome. In the case of transposons, these modifications will suppress recombination between transposon sequences, provide heritable, pre-transcriptional silencing of transposon-derived transcripts, and suppress transposon “jumping”. In the case of repeat sequences, there modifications will suppress recombination between repeat sequences.

In both cases, the outcome will be an enhancement of genomic stability. Such an outcome may be readily assessed by cytogenetic analysis, and by other means depending on therapeutic context. For example, where the method is being performed to enhance the genomic stability of cells of a pre-cancerous growth, the outcome may be measured as a failure of the pre-cancerous growth to become malignant.

### Genomic Imprinting and X-Inactivation

The methods of the present invention may also be used in therapeutic contexts to treat disorders of aberrant genomic imprinting or X-inactivation.

Genomic imprinting describes the phenomenon whereby a gene is specifically expressed only from the maternal (i.e., inherited from the mother) or paternal (i.e., inherited from the father) allele. This phenomenon results from specific silencing of the non-expressed paternal or maternal allele. Several human syndromes and disorders can result from a disruption of appropriate genomic imprinting (see, e.g. Yun. Histol Histopathol 1998;13:425; Tycko. Mutat Res 1997;386:131; Lalande. Annu Rev Genet 1996;30:173; Reeve. Med Pediatr Oncol 1996;27:470; and O'Dell and Day. Int J Biochem Cell Bio 1998;30:767).

X-inactivation describes the phenomenon whereby, in female mammals, one of the two X-chromosomes of each cell is inactivated so as to prevent expression of genes contained on that copy of the X chromosome. X-inactivation is mediated in cis by specific a non-coding RNA called Xist (Avner and Heard. Nat Rev Genet 2001; 2:59 and Sleutels et al. Nature 2002;415:810). Histone H3 lysine 9 methylation immediately follows the appearance of the non-coding Xist transcript, whose expression is regulated by an antisense RNA, Tsix and by promoter methylation (Heard et al. Cell 2001;107:727).

Several syndromes and disorders that result from aberrant genomic imprinting or X-inactivation have been identified. For example, the gene encoding insulin-like growth factor 2 (IGF2) is imprinted, with the paternal allele expressed and the maternal allele silenced. Loss of this imprinting, wherein the maternal allele of Igf2 is also expressed, has been demonstrated in several tumor types including Wilm's Tumor, in the Beckwith-Wiedemann syndrome of somatic overgrowth, and in gigantism (Yun. Histol Histopathol 1998;13:425; Reeve. Med Pediatr Oncol 1996;27:470; and O'Dell and Day. Int J Biochem Cell Bio 1998;30:767). In addition, in the general adult population a gene cluster containing the Igf2 gene may influence body weight, indicating that IGF2 expression may represent a therapeutic target for intervention in obesity (O'Dell and Day. Int J Biochem Cell Bio 1998;30:767).

The methods of the present invention provide for therapeutic intervention in such disorders, by providing the means to re-establish allele-specific heritable silencing of an improperly imprinted target locus or of a target locus on an improperly inactivated X-chromosome. In this context, it should be noted that the method of the present invention provides strong advantages over other methods in the art for reducing gene expression, because the gene silencing can be targeted to a specific allele of a target locus. For example, where the disorder or condition is associated with improper expression of the maternal allele of a gene, an RNAi probe that is complementary to a sequence specifically present within the maternal copy of a target locus spanning or adjacent to the gene, but which sequence is not found within the paternal copy of the target locus, may be used to specifically direct gene silencing of the maternal allele by directing assembly of the maternal allele of the gene into transcriptionally silent heterochromatin and/or by stimulating methylation of the maternal allele.

For example, in the case of Wilm's Tumor (a cancer of the kidney that primarily affects children, also known as nephroblastoma) or other cancers associated with improper expression of the maternal allele of Igf2, an RNAi probe that is complementary to a sequence present within the maternal copy of a target locus spanning or adjacent to the IgF2 gene, but absent within the corresponding paternal copy, may be introduced into a cell. This RNAi probe will then activate the RNAi machinery to assemble heterochromatin across, direct modification of histone on, and/or stimulate DNA methylation of the target locus. The maternal Igf2 allele will thereby be assembled into transcriptionally inactive heterochromatin, providing heritable, pre-transcriptional gene silencing of the maternal Igf2 allele.

The therapeutic outcome may be assessed by any of several techniques depending upon the nature of the imprinted gene and the syndrome and/or conditioned being addressed. For example, in the case of Wilm's Tumor, the treated cells may show a decreased rate of cell proliferation or a patient may show a reduction in tumor volume. In the case obesity or gigantism caused by expression of the maternal allele of Igf2, a patient may show reduced weight, reduced percent body fat, reduced height, reduced bone length, etc.

### Aneuploidy, Partial Aneuploidy, and Gene Duplications

The methods of the present invention may also be used in therapeutic contexts associated with disorders wherein the total number of copies of a target locus is altered. The total number of copies of a target locus in a cell may be altered, for example, by aneuploidy (e.g., wherein an entire extra chromosome is present as seen in trisomy 21, a.k.a. Down's syndrome), partial aneuploidy (e.g., wherein the cell contains an extra copy of a large segment of a chromosome as can occur as the result of translocation), or by gene duplication (e.g., as may be caused by unequal crossing over, such that a gene is tandemly duplicated). For example, gene duplications are a hallmark of certain cancers, wherein for example an endogenous oncogene is present in multiple copies (e.g., tandem duplication events responsible for amplification of a mutant RET gene is often observed in multiple endocrine neoplasia type 2 (MEN 2)-associated tumors).

The methods of the present invention provide the means to intervene in such disorders, by providing the means to establish allele-specific heritable silencing of an improperly duplicated target locus. As discussed above, the method of the present invention provides strong advantages over other methods in the art for reducing gene expression, because heritable silencing can be targeted to a specific allele of a target locus. For example, where the disorder or condition is associated with improper expression of a gene from within an extra copy or copies of a target locus, an RNAi probe that is complementary to a sequence specifically present within the extra copy or copies of the target locus, but which sequence is not found within at least two the other copies of the target locus (to provide the normal biallelic situation), may be used to specifically direct silencing of the extra copy or copies of a target locus, by directing assembly of the target locus into transcriptionally silent heterochromatin, directing modification of the histones on the DNA of the target locus, and/or by stimulating methylation of the DNA of the target locus.

The therapeutic outcome may be assessed by any of several techniques depending upon the nature of the syndrome and/or conditioned being addressed. For example, in the case of a cancer resulting from gene duplication, the treated cells may show a decreased rate of cell proliferation or a patient may show a reduction in tumor volume.

### Disorders of Chromatin Assembly and Genome Instability

The methods of the present invention may also be used in therapeutic contexts associated with disorders wherein the structure and/or function of chromatin is aberrant. In particular, the methods of the invention will be useful to treat disorders associated with decondensation of chromatin. For example, the heritable genetic disorder Ataxia Telangiectasia (AT) is characterized by significant decondensation of the nuclear chromatin in lymphoblastoid cells (Vergani et al. J Cell Biochem 1999;75:578). Chromatin decondensation can have severely deleterious consequences for the genomic stability of a cell, for example by causing aberrant centromere and telomere function and aberrant chromosome segregation during cell division, derepression of transposons and repeat elements, increased recombination rates, increased chromosome fragility, etc. These consequences may lead, for example, to decreased cell viability or to increased likelihood of the progression of the cell to neoplasm or malignant growth.

The methods of the present invention provide the means to intervene in such disorders, by providing the means to re-establish domains of condensed heterochromatin on decondensed centromeres, telomeres, transposons, repeat elements, etc. For example, an RNAi probe complementary to a given de-condensed target locus (e.g., centromeric or telomeric sequence, transposon or repeat element, etc.) may be introduced to a cell. The RNAi probe will then activate the RNAi machinery to assemble condensed heterochromatin across, and/or direct modification of histone on, the target locus sequences in their various locations throughout the genome. By promoting the assembly of the decondensed genomic regions into condensed heterochromatin, the method will attenuate the affects on genomic instability.

The therapeutic outcome may be assessed by any of several techniques depending upon the nature of the syndrome and/or conditioned being addressed. For example, in the case of Ataxia Telangiectasia (AT), RNAi probe-treated lymphoblastoid cells may show enhanced cell viability, decreased incidence of progression to neoplasm or malignant growth, or improved genomic stability as assessed by cytogenetic analysis.

### Experimental Models of Deletion Syndromes

Certain human disorders are due to the occurrence of chromosomal deletions of one or more genes and surrounding sequences. For example, the human developmental disorder known as DiGeorge/velo-cardio-facial syndrome is associated with a deletion of a region of chromosome 22 (22q11.2) (see, e.g., Bartsch et al. Am J Med Genet 2003;117A:1), while human cri du chat syndrome (CdCS) is associated with a deletion of the short arm of chromosome 5 (See, e.g., Mainardi et al. J Med Genet 2001;38:151).

The methods of the present invention will be useful to prepare experimental models of the human deletion syndromes by providing methods to assemble domains of chromosomes into an inactive conformation. For example, an RNAi probe complementary to a target locus within a chromosome region deleted in a deletion syndrome may be introduced to a cell. The RNAi probe will then activate the RNAi machinery to assemble condensed heterochromatin across, and/or direct modification of histone on, the target locus sequences. These regions of condensed heterochromatin and/or modified histone may then spread to encompass genes spanning and/or adjacent to the target locus.

These modifications in heterochromatin and/or histones will result in the silencing of the encompassed chromosomal domain. This silencing may serve to recapitulate the effects of large deletions on gene expression and gene regulation across a multi-gene domain of the chromosome. Thus, by promoting the assembly of the genomic regions spanning and surrounding the target locus into condensed heterochromatin, the introduction of the RNAi probe may be used to recapitulate the molecular consequences of a chromosome deletion.

The method may be used to develop in vitro cell culture models of deletion syndromes and/or whole animal models of deletion syndromes. Such experimental models may be used to characterize the molecular, cellular, and developmental consequences of deletion syndromes. For example, a transgenic mouse expressing or more RNAi probes complementary to a portion of the chromosome 22 region (22q11.2) commonly deleted in DiGeorge/velo-cardio-facial syndrome may serve as an experimental model for the human disorder. Such a mouse may show one or more of the malformations associated with DiGeorge/velo-cardio-facial syndrome, including heart defects, low set ears, cleft palate, etc.

### EXAMPLES

The following Examples illustrate the invention, but are not limiting.

In accordance with the present invention there may be employed conventional molecular biology, microbiology, protein expression and purification, antibody, and recombinant DNA techniques within the skill of the art. Such techniques are explained fully in the literature. See, e.g., DNA Cloning: A Practical Approach, Volumes I and II (Glover ed.:1985); Oligonucleotide Synthesis (Gait ed.:1984); Nucleic Acid Hybridization (Hames & Higgins eds.:1985); Transcription And Translation (Hames & Higgins, eds.:1984); Animal Cell Culture (Freshney, ed.:1986); Immobilized Cells And Enzymes (IRL Press: 1986); Perbal, A Practical Guide To Molecular Cloning (1984); Ausubel et al., eds. Current Protocols in Molecular Biology, (John Wiley & Sons, Inc.: 1994); Sambrook et al. Molecular Cloning: A Laboratory Manual, Third Edition (Cold Spring Harbor Laboratory Press: 2001); Harlow and Lane. Using Antibodies: A Laboratory Manual (Cold Spring Harbor Laboratory Press: 1999); PCR Primer: A Laboratory Manual, Second Edition. Dieffenbach and Dveksler, eds. (Cold Spring Harbor Laboratory Press: 2003); and Hockfield et al. Selected Methods for Antibody and Nucleic Acid Probes (Cold Spring Harbor Laboratory Press: 1993).

### Example 1

### Centromeric Silencing Depends on the RNA Interference (RNAi) Machinery

### Materials and Methods

Yeast strains: S. pombe deletion strains dcr1⁻, rdp1⁻, and ago1⁻ were generated in diploid cells by homologous recombination of a kanMX6 reporter gene flanked by gene specific sequences (as described in Bahler et al. Yeast 1998;14:943). Strains were confirmed by Southern blot and backcrossed 3 times before use in experiments.

The construction of S. pombe strains carrying the ura4⁺ transgene integrated into the central region of cen1 (cnt), into the right inverted repeat of cen1 (imr), and into the dg repeat of the right outermost region of cen1 (otr) and carrying the ura4-DS/E minigene at the endogenous location has been described (Allshire et al. Cell 1994;76:157 and Allshire et al. Genes & Devel 1995;9:218). In the ura4-DS/E minigene, 280 bp has been deleted from within the ura4 gene at its endogenous locus chromosome arm of chromosome 3. This deletion results in the ura4-DS/E minigene, which produces a truncated non-functional ura4 transcript.

Individual S. pombe strains carrying one of the centromeric ura4+ transgene integrations as well as the chromosome arm ura4-DS/E minigene were mated to each of the S. pombe ago1⁻, dcr1⁻ and rdp1⁻ deletion strains, to generate strains carrying a centromeric ura4⁺ transgene, the ura4-DS/E minigene, and an RNAi machinery gene deletion.

Generation of the S. pombe strain swi6-115 (a.k.a. swi6⁻), in which the swi6 gene is mutated, has been described (See, e.g., Egel et al. Proc Natl Acad Sci US 1984;81:3481 and Thon and Klar. Genetics 1993;134:1045).

S. pombe strains carrying both the ago1 deletion and the swi6-115 mutation (ago1⁻ swi6) were obtained by mating the single mutant strains.

Iodine staining assessment of mating-type switching: An iodine-staining procedure is used to estimate the efficiency of mating-type switching in S. pombe. Individual colonies grown on sporulation medium were exposed to iodine-vapors. Synthesis of a starch-like compound by sporulating cells results in black staining of colonies after exposure to iodine vapors (Bresch et al. Mol Gen Genet 1968;102:301). In efficiently switching strains, homogenous distribution of the cells of the two different mating types leads to efficient mating and sporulation, and hence colonies stain darkly with iodine vapors. In contrast, strains that switch inefficiently stain lightly with iodine vapors due to poor mating and sporulation.

Purification of RNA: Total RNA was extracted from yeast cells essentially as described in Tyers et al. EMBO J. 1992;l 1:1773. Briefly, mid-log phase yeast cultures were vortexed in LETS buffer (100 mM LiCi, 10 mM EDTA, 10 mM Tris-HCl pH 7.4, and 0.2% SDS), phenol, and glass beads. RNA was then extracted twice with phenol chloroform and precipitated in 1/10 volume 5M LiCl and 2.5 volumes of ethanol.

Northern blot analysis of purified RNA: For Northern analysis, RNA was purified from mid-log phase cultures of S. pombe ago1⁻, dcr1⁻, rdp1⁻, swi6−, and ago1⁻ swi6⁻ strains and wildtype S. pombe. Northern analysis of the purified RNA was performed as described in Tyers et al. EMBO J. 1992;11:1773. Briefly, purified total RNA samples were resolved on 1% agarose gels containing 6.7% formaldehyde, blotted to nylon membranes, cross-linked, and hybridized with radiolabeled probes specific for act1 (actin), ura4, or centromeric otr region dg repeats.

The act1 hybridization probe was composed of the nucleotide sequence found at Genbank Accession # Y00447, while the ura4 hybridization probe was composed of the nucleotide sequence found at Genbank Accession # X13976. The centromeric otr region dg repeat hybridization probe sequences were isolated by PCR performed using the following primers: 5′-GTT CAG CTG GGA TGG ATG AT-3′ (SEQ ID NO: 1) and 5′-CCC TAA CTT GGA AAG GCA CA-3′ (SEQ ID NO: 2).

Note that that the ura4⁺ transgene and ura4-DS/E minigene transcripts are detected the same ura4 probe, but vary slightly in size. Detection of act1 expression using the act1 probe served as a positive control. Radiolabel signal of hybridized probes was detected using a FUJI phosphoimager.

Strand-specific RT-PCR analysis of purified RNA: For strand-specific RT-PCR analysis, mid-log phase cultures of S. pombe ago1⁻, dcr1⁻, rdp1⁻, swi6−, and ago1⁻ swi6⁻ strains and wildtype S. pombe were grown in yeast extract supplemented with adenine (YEA) at 32° C. RNA was then extracted as described above and the purified RNA was treated with DNase (RQ1, Promega). This RNA was then analyzed by RT-PCR (OneStep RT-PCR kit, Qiagen), performed essentially according to the manufacturer's instructions. Briefly, RNA samples were incubated with a single primer, complementary to either the forward or reverse centromeric transcript, in first strand cDNA synthesis reactions. The primers used for this reaction, cen FOR (5′-GAA AAC ACA TCG TTG TCT TCA GAG-3′, SEQ ID NO: 3) and cen REV (5′-CGT CTT GTA GCT GCA TGT GAA-3′, SEQ ID NO: 4) are complementary to either the forward or reverse transcript derived from the centromeric otr region dh repeats. The reverse transcriptase was then inactivated for 15 min at 95° C. The second primer was then added so that both primers were present in subsequent cycles of PCR amplification. Treatment of control reactions lacking reverse transcriptase (-RT) was identical, except these samples were not subjected to first strand synthesis. These -RT reactions served as the negative control. As a positive control, strand-specific RT-PCR reactions were also conducted using primers specific for act1 sense (primer act1 s) or act1 antisense (primer act1 as) transcripts. The act1 nucleotide sequence may be found at Genbank Accession # Y00447.

Nuclear run-on assays of purified RNA: Nascent RNA from mutant and wildtype fission yeast strains was analyzed by nuclear run-on assay. For this assay, S. pombe ago1⁻, dcr1⁻, rdp1⁻, swi6−, and ago1⁻ swi6⁻ strain and wildtype S. pombe cells were grown to mid-log phase in yeast extract supplemented with adenine (YEA) at 32° C. The cells were filtered and washed in ice-cold TMN buffer (10 mM Tris, pH 7.4, 100 mM NaCl, 5 mM MgCl₂). The cells were allowed to equilibrate in the TMN buffer for 10 min on ice. The TMN buffer was then removed and the cells incubated 0.5% sarkosyl on ice for 20 min. The permeabilized cells were then resuspended in run-on buffer (50 mM Tris-HCl, pH7.9, 5 mM MgCl₂, 1 mM MnCl₂, 100 mM KCl, 2 mM DTT, 0.5 mM rATP, 0.25 mM rGTP, 0.25 mM rCTP, 10 mM phosphocreatine, 12 μg/ml phosphocreatine kinase, 100 units of RNase inhibitor (Roche), and 100 μCi rUTP-alphaP³² (Amersham # PB10203 or #AA0003)) and incubated at 25° C. for 8 min. Labeled RNA was extracted twice with TMN equilibrated Phenol/Chloroform and hybridized to nylon membranes containing strand specific primers for the centromeric otr repeats (cen FOR and cen REV, as described above). Hybridization to strand specific primers for act1 (act1 s and act1 as, as described above) served as a positive control.

Sequencing of centromeric repeat transcripts in RNAi machinery mutant yeast: For sequence analysis, RNA was purified from mid-log phase cultures of S. pombe ago1⁻ deletion strains. Next, sequences for the centromeric repeat transcripts were amplified by 5′ RACE PCR performed using the “5′ RACE system for rapid amplification of cDNA ends, version 2.0” kit (Invitrogen). 5′ RACE was performed according to the manufacturer's instructions using nested primers for the centromeric otr region dh repeat (5′-GCT TTA TGC CAA AAC ATG CA-3′, SEQ ID NO: 5; and 5′-TGC ATG TTT TGG CAT AAA GC-3′, SEQ ID NO: 6) and nested primers for the centromeric otr region dg repeat (5′-AAC CAA CGA CAT CAT GGG TAG-3′, SEQ ID NO: 7; and 5′-CTA CCC ATG ATG TCG TTG GTT-3′, SEQ ID NO: 8).

The RACE PCR products (150-600 bp) were then cloned into TOPO-TA vectors (Invitrogen) and sequenced from both ends according to methods well established in the art. Sequence comparisons conducted using the BLAST algorithm were used to determine the centromere (cen1, cen2, or cen3) from which each product was derived. This assignment was unambiguous for dh repeat sequences (i.e., the centromere of origin could be determined), but not for dg repeat sequences, which are highly conserved across all repeats at all three centromeres.

### Results and Discussion

The RNAi machinery genes ago1⁺, dcr1⁺ and rdp1⁺ were deleted by homologous gene replacement in diploid strains of the fission yeast S. pombe, and the ago1⁻, dcr1⁻ and rdp1⁻ mutants were found to be viable as haploids. Mating and mating-type switching was not affected in ago1⁻ as assessed by iodine staining and backcrossing. However, trans-acting mutants that affect centromeric silencing without affecting the mating-type locus had been previously isolated in S. pombe (Ekwall et al. Genetics 1999; 153:1153). Therefore the effect of these three mutations on centromeric silencing was tested.

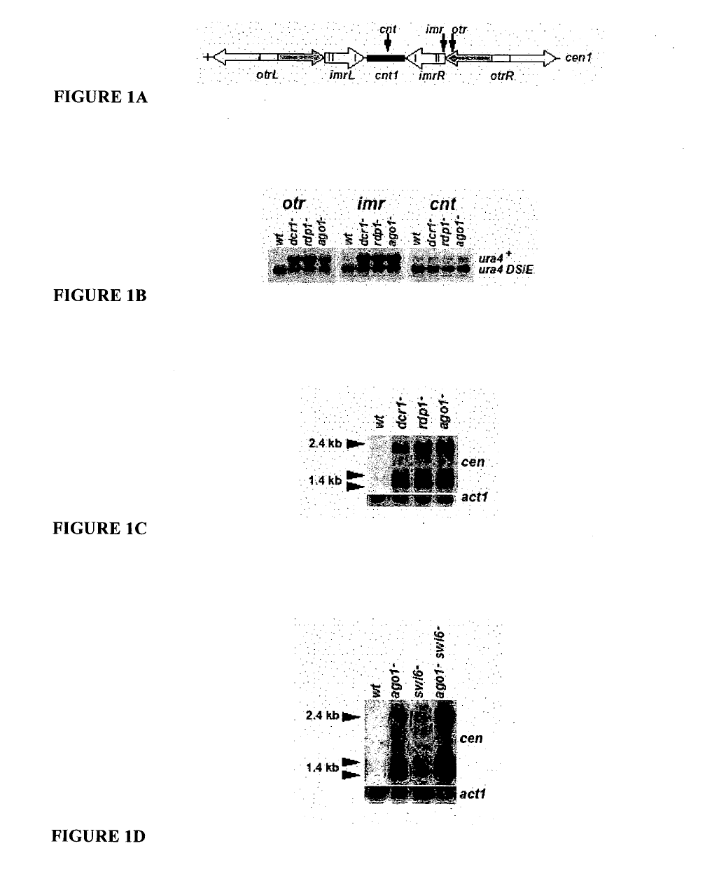

The S. pombe genome contains three centromeres: cen1, cen2, and cen3. The central region of each centromere (cnt) is flanked by large inverted repeats (imr) containing tRNA genes, and then by the outermost region (otr) which is comprised of tandem alternating copies of dg and dh repeats (also known as K repeats) (Takahashi et al. J Mol Biol 1991;218:13 and Steiner et al. Mol cell Biol 1993;13:4578). See FIG. 1A for a diagram of the structure of cen1.

For this study, the ura4⁺ transgene was introduced into three different positions within the cen1 region of S. pombe. One transgene was integrated into the central region of cen1 (cnt), one was integrated into the right inverted repeat of cen1 (imr), and one was integrated into the dg repeat of the right outermost region of cen1 (otr) (see FIG. 1A). As a control, each strain also contained the ura4-DS/E mini-gene, containing a 280 bp deletion at the endogenous ura4 locus on the chromosome arm of chromosome 3.

It has previously been shown that ura4⁺ transgenes integrated in each of these centromeric regions on chromosome 1 (cnt, imr, and otr) are silenced in wildtype strains (Allshire et al Cell 1994;76:157 and Allshire et al. Genes Dev 1995;9:218) while transcription of a ura4-DS/E minigene located on the chromosome arm is unaffected (Allshire et al. Cell 1994;76:157).

Individual S. pombe strains carrying one of the ura4⁺ transgene integrations (cnt, imr, or otr) and the ura4-DS/E minigene were mated to each of the S. pombe ago1⁻, dcr1 and rdp1 deletion strains, to generate strains carrying a centromeric ura4⁺ transgene, the ura4-DS/E minigene, and an RNAi machinery gene deletion.

Silencing of the centromeric transgenes was then assessed via Northern blot analysis of transcription of the ura4⁺ transgene. Transcription of the ura4-DS/E minigene served as an internal control. Each of the two ura4⁺ transgenes located centromere distal to the tRNA genes (imr and otr) were de-repressed in the ago1⁻, dcr1⁻ and rdp1⁻ S. pombe strains, as shown by high level expression of the transgene mRNA (FIG. 1B). However, the ura4⁺ transgene located within the central region (cnt) remained silenced (FIG. 1B). Similar results were obtained in all 3 mutant strains.

In Drosophila, the ARGONAUTE homolog sting/aubergine is responsible for processing a heterochromatic RNA, Stellate, from the Y chromosome (Schmidt et al. Genetics 1999;151:749 and Aravin et al. Curr Biol 2001; 11:1017). It was next tested whether the centromeric repeats themselves were transcribed in the fission yeast RNAi mutants ago1⁻, dcr1 and rdp1. It was also tested whether these centromeric repeats were transcribed in swi6 mutants, and in ago1 swi6 double mutants.

Transcripts derived from cen repeats were not observed in wildtype strains in agreement with previous reports (Fishel et al. Mol Cell Biol 1988;8:754; see FIGS. 1C and 1D). However, three major transcripts that hybridized to the cen repeats were found to accumulate at high levels in each of the RNAi mutants (cen, FIG. 1C). These transcripts were also found in swi6 mutants (cen, FIG. 1D), but at a much lower level. The ago1⁻ swi6⁻ double mutants had higher levels of transcript than swi6⁻ single mutants (FIG. 1D), indicating Ago1 could function in the absence of Swi6, and likely acted upstream.

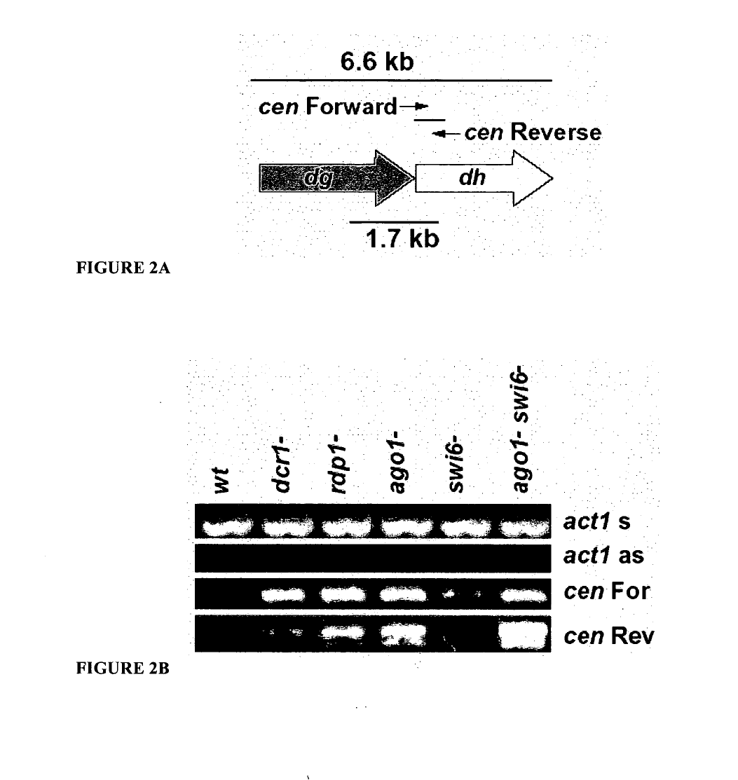

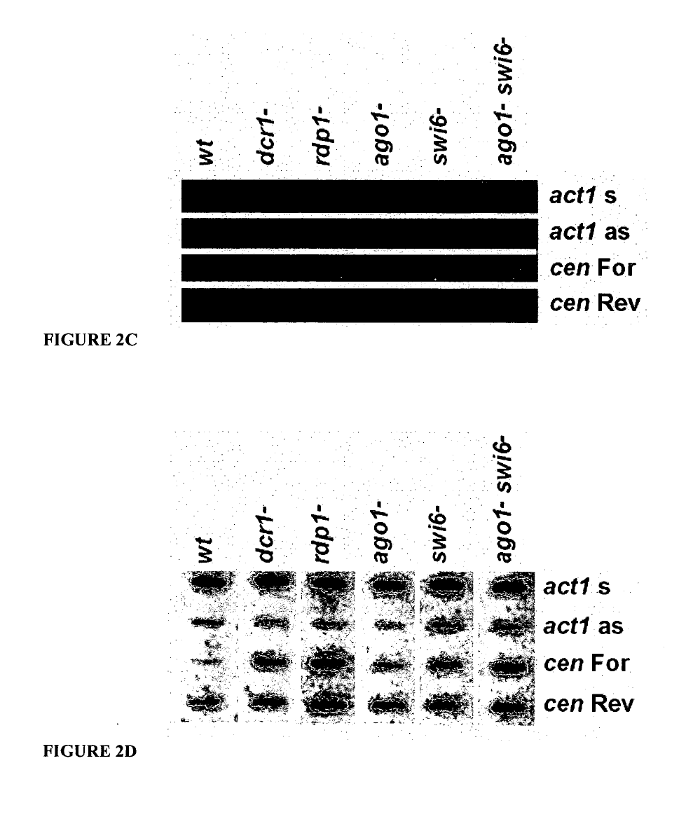

It was next determined from which strand of the centromeric repeat the observed transcripts were transcribed (FIG. 2A), using strand specific RT-PCR (reverse transcriptase-polymerase chain reaction). This assay was performed in the presence of reverse transcriptase (FIG. 2B), or in the absence of reverse transcriptase as a negative control (FIG. 2C). The observed centromeric transcripts were derived from both strands in each of the RNAi mutants, but only from one strand (which was designated as the forward transcript) in swi6⁻ mutants (FIG. 2B). In the case of the ago1⁻ swi6⁻ double mutant, centromeric transcripts were derived from both strands (FIG. 2B). In wildtype cells the forward transcript did not accumulate (FIG. 2B), although very low levels of reverse transcripts were detected when the number of PCR cycles was increased.

In order to test whether the appearance of these transcripts in the RNAi mutants reflected a change in transcriptional or post-transcriptional regulation, run-on transcription experiments were performed. RNA was purified from nuclei that had been permeabilized with detergents to inhibit transcriptional initiation while allowing incorporation of radioactive UTP. This RNA was hybridized to slot-blots of strand specific primers for both the actin gene and the centromeric repeats (FIG. 2D).

Nascent forward transcripts were detected in all mutant strains, but not in wildtype strains (FIG. 2D). This indicates that appearance of the forward transcripts in the mutants (FIG. 2B) reflects an alteration in transcriptional control, presumably regulated via Swi6. In contrast, nascent reverse transcripts were detected in both mutant and wildtype nuclei (FIG. 2D), but only accumulated in the RNAi mutants, and not in swi6 mutant (FIG. 2B).

These results show that the reverse strand of the centromeric repeat is always transcribed in wildtype cells, but that this transcribed RNA is rapidly turned over by the RNAi machinery. The forward strand is not transcribed in wildtype cells, but is transcribed in the RNAi mutants, indicating the RNAi machinery represses forward transcription indirectly.

The sequences of portions of the centromeric repeat transcripts from S. pombe ago1⁻ cells were identified in order to determine which centromeres (cen1, cen2, and/or cen3) were subject to transcriptional and post-transcriptional regulation by the RNAi machinery. The results of this sequence analysis indicated that transcripts of centromeric repeats from all 3 centromeres were present in S. pombe ago1⁻ cells, indicating that all three centromeres are coordinately regulated by the RNAi machinery.

### Example 2

### The RNAi Machinery is Required for Histone H3 Lysine-9 Methylation

### Materials and Methods

Chromatin immunoprecipitation (ChIP) analysis: ChIP analysis was performed essentially as described in Nakayama et al. Science 2001;292: 110 and Nakayama et al. Cell 2000;101: 307. These assays used S. pombe wildtype (wt), ago1⁻, dcr1⁻, rdp1⁻, swi6−, and ago1⁻ swi6⁻ strains containing both the ura4⁺ transgene inserted into the dg repeat of the right outermost region of cen1 (otr) and the ura4-DS/E minigene on the chromosome arm of chromosome 1. Yeast cells were grown to mid-log phase in yeast extract supplemented with adenine (YEA) at 32° C. Cultures were then shifted to 18° C. for two hours. The cells were then fixed in 3% paraformaldehyde. Extracts were sonicated in order to fragment the genomic DNA to 0.5 to 0.8 kilobases (kb) in length. The fragmented DNA was then purified from whole cell extracts (wce), or immunoprecipitated with antibodies against Swi6 (Nakayama et al. Cell 2000;101: 307), histone H3 methyl-K4, histone H3 methyl-K9 (Nakayama et al. Science 2001;292:110; Noma et al. Science 2001;293:1150; and Upstate Biotechnology), histone H3 branched methyl-K9 (Peters et al. Cell 2001;107:323), or the 3×HA epitope (12CA5; available from the Cold Spring Harbor Laboratories monoclonal antibody facility).

DNA purification and immunoprecipitation were performed according to methods well established in the art (see, e.g., Sambrook et al. Molecular Cloning: A Laboratory Manual, Third Edition (Cold Spring Harbor Laboratory Press: 2001); Ausubel et al., eds. Current Protocols in Molecular Biology (John Wiley & Sons, Inc.: 1994); and Harlow and Lane. Using Antibodies: A Laboratory Manual (Cold Spring Harbor Laboratory Press: 1999).

This purified whole cell extract DNA and immunoprecipitated DNA was then analyzed by PCR using primers specific for the ura4⁺ transgene and ura4-DS/E minigene (5′-TTT GTG GCA TAA CAA GTT CTC AA-3′, SEQ ID NO: 9; and 5′-AAA CGA CCA ATA TGC TGC G-3′, SEQ ID NO: 10), the actin (act1) gene (5′-CCG CGG TCT TCT TCC GTG CGC-3′, SEQ ID NO: 11; and 5′-GCA AGA ATG GAT CCA CCA ATC C-3′, SEQ ID NO: 12), or the centromeric otr region dg repeats (5′-GCA ATG TTT TGC CAA AGC GAA ATT G-3′, SEQ ID NO: 13; and 5′-TCC AAG ACT GTT GTT GAG TGC TGT GGA-3′, SEQ ID NO: 14). Note that the ura4 primers amplify differently sized products from the ura4⁺ otr transgene and the ura4-DS/E minigene. As a negative control (−), PCR was performed on mock reactions without immunoprecipitated DNA. PCR was used to incorporate a radiolabel into the products. These labeled products were then resolved on 4% polyacrylamide gels or 1% agarose gels, and ethidium stained or visualized using a FUJI phosphoimager.

Quantitative PCR analysis of DNA from ChIP analysis: Purified DNA from whole cell extracts and DNA immunoprecipitated in ChIP experiments, using antibodies raised against the histone H3 methyl-K4, histone H3 methyl-K9, or histone H3 branched methyl-K9 peptides, were analyzed by real-time PCR using the SYBR Green Universal Mix for PCR and an ABI Prism 7700 (Perkin Elmer Applied Biosystem). Real time PCR was performed using primers specific for the actin (act1) gene (5′-CCG CGG TCT TCT TCC GTG CGC-3′, SEQ ID NO: 15; and 5′-GCA AGA ATG GAT CCA CCA ATC C-3′, SEQ ID NO: 16), the S. pombe matK mating type repeat, or the centromeric otr region dg repeats (5′-GCA ATG TTT TGC CAA AGC GAA ATT G-3′, SEQ ID NO: 17; and 5′-TCC AAG ACT GTT GTT GAG TGC TGT GGA-3′, SEQ ID NO: 18).

The output value obtained from real-time PCR analysis, Ct, is defined as the PCR cycle number that crosses an arbitrarily placed signal threshold and is a function of the amount of target DNA present in the starting material. Quantification was determined by applying the 2^{-Ct} formula and calculating the average of the three values obtained for each experimental sample divided by the average value of the corresponding control. A similar formula was used to subtract residual amplified DNA found in mock reactions without immunoprecipitated DNA. Values for the immunoprecipitated DNA from the different mutant and wt strains (experimental) were calculated relative to actin (act1⁺) for anti-H3 methyl-K4 immunoprecipitated DNA, or relative to the matK mating type repeat (K9) for anti-H3 methyl-K9 or anti-H3 branched methyl-K9 immunoprecipitated DNA (controls).

### Results and Discussion

S. pombe heterochromatin is marked by methylation of histone H3 at lysine 9 (K9), while methylation of lysine 4 (K4) is preferentially associated with expressed genes (Nakayama et al. Science 2001; 292:1110 and Noma et al. Science 2001;293: 1150).

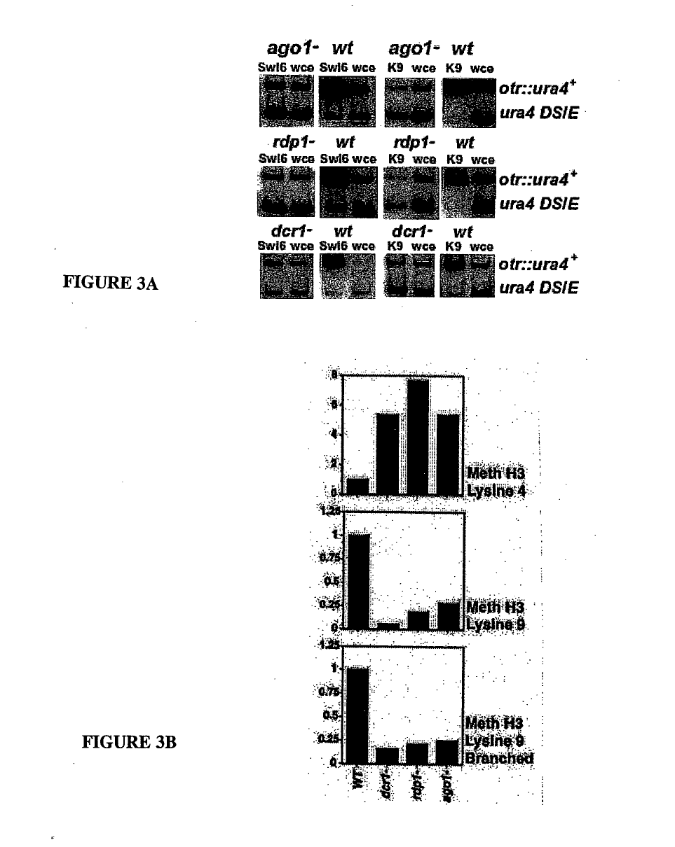

Therefore, it was tested whether K9 and K4 methylation of histone H3 were affected in S. pombe RNAi machinery mutants, using antibodies specific for each modification in chromatin immunoprecipitation (ChIP) experiments. In these experiments, the genomic DNA from formaldehyde fixed mutant or wildtype cells was fragmented, and then immunoprecipitated with antibodies raised against histone H3 methyl-K4, histone H3 methyl-K9, or branched histone H3 methyl K9. These immunoprecipitated DNAs were then PCR amplified using primers specific for centromeric dg repeats. Primers specific to the actin gene (act1), and primers specific for the or the K-repeat found at the mating type locus (Grewal and Klar. Genetics 1997;146:1221), which is relatively unaffected in the three RNAi machinery mutant strains, were used as controls. In some cases “real time” quantitative PCR was performed to quantitate the amount of centromeric repeat DNA found in the chromatin immunoprecipitations (FIG. 3A).

These results showed that both histone H3 modifications, methyl-K4 and methyl-K9, were associated with centromeric repeats in wildtype cells. In contrast, the RNAi machinery mutant dcr1⁻, rdp1⁻ and ago1⁻ cells had increased levels of H3 methyl-K4 in the centromeric region (FIG. 3A). In contrast, levels of H3 methyl-K9 were greatly reduced in the RNAi machinery mutant dcr1⁻, rdp1⁻ and ago1⁻ cells (FIG. 3A).

Among the three mutants, ago1⁻ retained significantly more H3 K9 methylation than dcr1⁻ (FIG. 3A). This observation was investigated further by repeating the chromatin immunoprecipitation using antibodies raised against branched histone H3 peptide tails methylated on K9. These antibodies detect closely packed, condensed modified nucleosomes in mammalian cells (Peters et al. Cell 2001;107:323). All 3 RNAi machinery mutants had similarly low levels of modification detected with these antibodies (FIG. 3A).

The pattern of histone H3 modification associated with a ura4⁺ transgene integrated in the outer region of cen1 (transgene otr, see FIG. 1A) was next examined. In these ChIP experiments, the fragmented DNAs were immunoprecipitated with antibodies raised against Swi6 and histone H3 methyl-K9, and the immunoprecipitated DNAs were PCR amplified using primers specific the ura4⁺ transgene and ura4-DS/E minigene.

The ura4⁺ transgene was associated with H3 methyl-K9 in wildtype cells, but this association was markedly reduced in each of the three RNAi machinery mutants (FIG. 3B). Swi6 depends on histone modification for chromatin binding. Thus, levels of Swi6 localization at the ura4⁺ transgene was also markedly reduced in each of the three RNAi machinery mutants, relative to wildtype cells (FIG. 3B). These changes in histone modification are consistent with the observed loss of silencing of the ura4⁺ transgene at this location (otr, see FIG. 1B). However, silencing of the ura4⁺ transgene was largely retained when it was integrated in the central portion of cen1 (cnt, see FIG. 1B). This different likely reflects the replacement of histone H3 with the histone variant cenpA in this region of the centromere (Takahashi et al. Science 2000;288:2215), as cenpA lacks an equivalent to H3 lysine 4 and lysine 9.

These results show that all three RNAi machinery mutants show a loss of closely packed K9 modified nucleosomes from the centromeric repeats and from a ura4⁺ transgene integrated nearby on cen1.

As discussed above, in S. pombe, heterochromatin is marked by methylation of histone H3 at lysine 9, while analogously in mammals, heterochromatin is marked by methylation of histone H3 at the lysine 9 and/or lysine 27 residue. Thus, the results reported for the effects of RNAi on heterochromatin and histone methylation in S. pombe cells constitute useful analogs by which to understand the effects of RNAi on heterochromatin and histone methylation in mammalian cells.

### Example 3

### The Role of RNA Dependent RNA Polymerase (Rdp1) and siRNA in Targeting Heterochromatin

### Materials and Methods

Yeast strains: S. pombe expressing triple HA-tagged Dcr1 (dicer) and Rdp1 (RNA dependent polymerase) proteins were generated by replacing the endogenous dcr1 and rdp1 genes with dcr1-3xHA and rdp1-3xHA sequences, respectively. The replacement was generated in diploid cells by homologous recombination of a dcr1-3xHA of rdp1-3xHA sequence plus a kanMX6 reporter gene construct flanked by gene specific sequences (as previously described in Bahler et al. Yeast 1998;14:943).

Strains were confirmed by Southern blot and backcrossed 3 times before use in experiments. Tagged strains were tested for expression of the tagged proteins by western blot using an antibody to the HA tag. Northern blot analysis (performed as described in Example 1) failed to detect expression of centromeric transcripts in dcr1-3xHA and rdp1-3xHA strains, indicating that the HA tag is not inhibiting the function of these fusion proteins.

Chromatin immunoprecipitation (ChIP) analysis: ChIP analysis was performed essentially as described in Example 2. For these assays, wildtype S. pombe cells were used. Fragmented DNA was then purified from whole cell extracts (wce), or immunoprecipitated with antibodies against the triple-HA tag (available from the Cold Spring Harbor Laboratories monoclonal antibody facility).

The purified whole cell extract DNA and immunoprecipitated DNA was then analyzed by PCR using primers specific for the centromeric otr region dg repeats (5′-GCA ATG TTT TGC CAA AGC GAA ATT G-3′, SEQ ID NO: 19; and 5′-TCC AAG ACT GTT GTT GAG TGC TGT GGA-3′, SEQ ID NO: 20). As a negative control (−), PCR was performed on mock reactions without immunoprecipitated DNA. The products were then resolved on 4% polyacrylamide gels or 1% agarose gels, and ethidium stained.

### Results and Discussion

If RNA interference is responsible for chromatin modification at the centromere, then components of the RNAi machinery might be expected to interact with centromeric sequences. To test this possibility, triple HA-tagged Dcr1 and Rdp1 proteins were immunoprecipitated from chromatin extracts of wildtype S. pombe cells. Chromatin immunoprecipitation (ChIP) was performed on extracts derived from S. pombe strains expressing 3×HA tagged Rdp1 or 3×HA tagged Dcr1, and from wildtype S. pombe (untagged Rdp1 and Dcr1). DNA fragments from whole cell extracts and DNA immunoprecipitated with antibodies raised against the triple-HA tag were amplified by PCR using centromere specific primers. As negative control, PCR was performed on a mock reactions without immunoprecipitated DNA.

In this experiment, centromeric repeats could be selectively amplified from HA-tagged Rdp1 immunoprecipitates, but not from Dcr1 tagged immunoprecipitates. This result indicates that that Rdp1, but not Dcr1, is bound to centromeric chromatin.

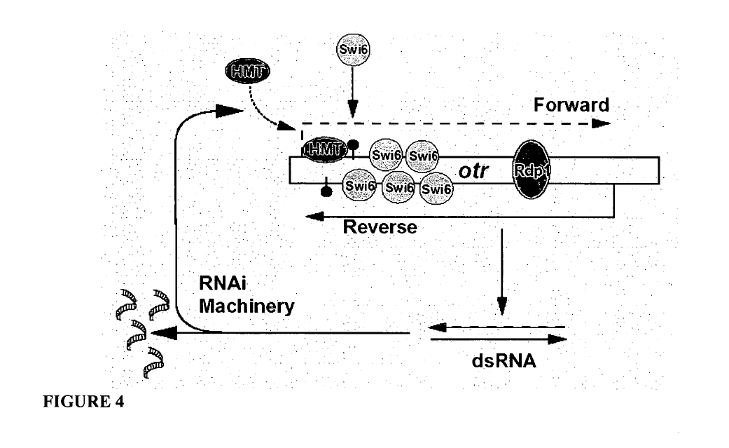

Although not intending to be limited by mechanism, the discoveries exemplified by Examples 1-3 are consistent with the following model for the role of the RNAi machinery in the initiation and maintenance of the heterochromatic state of centromeric repeats in the fission yeast S. pombe. In wildtype cells, reverse strand transcripts are initiated in centromeric otr region repeats on each chromosome arm, and transcribed towards the centromere (FIG. 4). However, these transcripts are rapidly turned over by the RNAi machinery. Secondary structure predictions failed to detect hairpin structures that might trigger cleavage. Instead, occasional transcription from the forward strand of cen1 could generate centromeric repeat dsRNA, triggering processing into siRNAs. The centromeric repeat dsRNA could then be continually regenerated by priming the reverse transcript with Rdpl enzyme bound to the chromatin. The resulting amplified heterochromatic siRNA then guides histone modification by histone methyltransferases (HMT), which would in turn recruit swi6⁺, thus pre-transcriptionally silencing expression of the forward strand (FIG. 4).

In mammals, higher order structure in pericentromeric heterochromatin has recently been shown to involve histone H3 K9 modification and an RNase-sensitive component found in total cellular RNA (Maison et al. Nat Genet. 2002;30:329). Thus it is considered that the RNAi machinery in heterochromatin formation observed for S. pombe is highly conserved, and operates in mammals as well.

### Example 4

### Swi6-Dependent Maintenance of Histone H3 Lys9 Methylation

### Materials and Methods

Yeast strains: Generation of the S. pombe strain swi6-115 (swi6⁻), in which the swi6 gene is mutated, has been described (see, e.g., Egel et al. Proc Natl Acad Sci US 1984;81:3481).

Generation of the S. pombe strain Kint2::ura4⁺, in which the ura4⁺ transgene is inserted at the cenH region between mat2P and mat3M of the S. pombe mating type region, has been described (see, e.g., Grewal and Klar. Genetics, 1997;146:1221). As used herein, this strain also contains the ura4-DS/E mini-gene on the chromosome arm of chromosome 3 (see Example 1).

Generation of the S. pombe strain KΔ::ura4⁺, in which the cenH-containing region between mat2P and mat3M of the S. pombe mating-type region is replaced by the ura4⁺ transgene, has been described (see, e.g., Thon and Klar. Genetics 1993;134:1045; Grewal and Klar. Cell 1996;86:95, and Grewal and Klar. Genetics, 1997;146:1221). As used herein, this strain also contains the a ura4-DS/E mini-gene on the chromosome arm of chromosome 3 (see Example 1).

Cells of S. pombe strain KΔ::ura4⁺ that had epigenetically silenced the ura4⁺ transgene (ura4-off) were identified as cells which were able to grow in uracil supplemented media containing 5-fluoroorotic acid (FOA), but were unable to grow in uracil depleted media. Conversely, cells of S. pombe strain KΔ::ura4⁺ that had failed to epigenetically silence the ura4⁺ transgene (ura4-on) were identified as cells which were unable to grow in uracil supplemented media containing 5-fluoroorotic acid (FOA), but were able to grow in uracil depleted media.

Cells of S. pombe strain KΔ::ura4⁺ that had epigenetically silenced the tightly linked endogenous his2 gene (his2⁻) were identified as cells which were able to grow in histidine supplemented media, but were unable to grow in media lacking histidine. Conversely, cells of S. pombe strain KΔ::ura4⁺ that had not epigenetically silenced the tightly linked endogenous his2 gene (his2⁺) were identified as cells which were able to grow in media lacking histidine.

Generation of S. pombe Kint2::ura4⁺ and KΔ::ura4⁺ strains that also contain the swi6-115 mutation, and are therefore swi6, has been described (see, e.g., Egel et al. Proc Natl Acad Sci US 1984; 81:3481; Thon and Klar. Genetics 1993; 134:1045; Grewal and Klar. Cell 1996; 86:95; and Grewal and Klar. Genetics, 1997; 146:1221).

The S. pombe strain swi6⁺-333, in which three copies of the swi6 coding sequences were inserted at the endogenous swi6 chromosomal location was constructed as previously described (Nakayama et al. Cell 2000;101:307).

The S. pombe strain swi6⁺-333 was then mated to cells of S. pombe strain KΔ::ura4⁺ that had failed to epigenetically silence the ura4⁺ transgene (ura4-on) to generate cells that contained both the swi6⁺-333 allele of swi6 and the ura4-on epiallele of KΔ::ura4⁺.

The S. pombe strain clr4⁺-666, in which six copies of the clr4 coding sequences were inserted at the endogenous clr4 chromosomal location was constructed using standard techniques well known in the art.

The S. pombe strain clr4⁺-666 was then mated to cells of S. pombe strain KΔ::ura4⁺ that had failed to epigenetically silence the ura4⁺ transgene (ura4-on) to generate cells that contained both the clr4⁺-666 allele of clr4 and the ura4-on epiallele of KΔ::ura4⁺.

Tetrad analysis: Mating, sporulation and tetrad analysis of S. pombe strains was performed as previously described (see, for example, Moreno et al. Methods Enzymol 1991;194:795).

Chromatin immunoprecipitation (ChIP) analysis: ChIP analysis was performed as described in Example 2. For these assays, Kint2::ura4⁺ and KΔ::ura4⁺ S. pombe strains that were wildtype for swi6 (swi6⁺) or that contained the swi6-115 mutation (swi6⁻) were used. Fragmented DNA was then purified from whole cell extracts, or immunoprecipitated with the antibodies against Swi6 or histone H3 methyl-Lys9.

This purified whole cell extract DNA and immunoprecipitated DNA was then analyzed by PCR using primers specific for the ura4⁺ transgene and ura4-DS/E minigene (See Example 2). Note that this is a competitive PCR strategy, whereby one primer-pair amplifies different size PCR products from the full-length ura4⁺ located at respective Kint2::ura4⁺ or KΔ::ura4+locations, and from the control ura4DS/E minigene. The PCR was used to incorporate a radiolabel into the products. These labeled products were then resolved on 4% polyacrylamide gels and visualized and quantitated using a FUJI phosphoimager.

The ratios of ura4⁺ and ura4DS/E signals in the experimental (immunoprecipitated DNA) versus control (DNA from whole cell extracts) conditions were used to calculate the relative precipitated fold enrichment of associated Swi6 or histone H3 methyl-K9 proteins in strains that were wildtype for swi6 (swi6⁺) or that contained the swi6-115 mutation (swi6).

### Results and Discussion

The role of Swi6 in maintenance of H3 Lys9-methylation was examined in another model for heterochromatin, the silenced mating-type region. As discussed above, previous findings have suggested a model for heterochromatin formation in which the cooperative activity of HDACs and the H3 Lys9 methyltransferase Clr4 establish a “histone code” that is essential for the localization of Swi6 to silenced genomic locations (Nakayama et al. Science 2001;292:1110). It has also been shown that when inserted into the silenced mating-type region as a Kint2::ura4⁺ transgene, the ura4⁺ sequences are associated with Lys9-methylated histone H3 and with Swi6 protein (Nakayama et al. Science 2001; 292: 110).

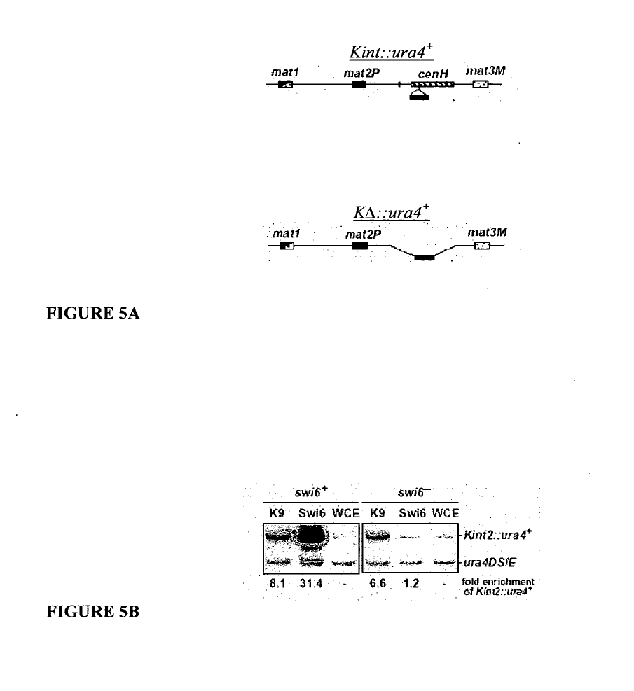

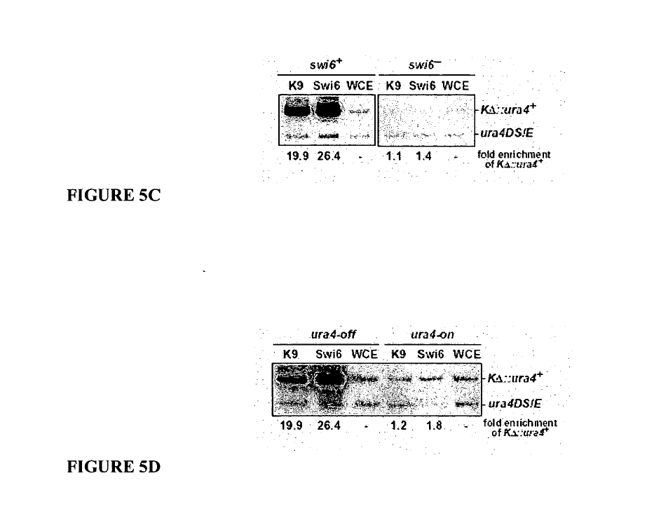

These studies detected the association of Lys9-methylated histone and Swi6 protein with the ura4⁺ transgene sequences in Kint2::ura4⁺ (FIGS. 5A and 5B) and KΔ::ura4⁺ (FIGS. 5A and 5C) S. pombe strains that were wildtype for swi6 (swi6⁺) or that contained the swi6-115 mutation (swi6⁻).

Although H3 Lys9 methylation is required for the chromatin association of Swi6, mutations in Swi6 have minimal effects on levels of H3 Lys9 methylation at a Kint2::ura4⁺ reporter gene inserted within the cenH repeat (FIG. 5B), indicating that H3 Lys9 methylation acts upstream of Swi6. However, in KΔ::ura4⁺ cells lacking the cenH repeat, the presence of H3 Lys9 methylation strictly depends on Swi6 (FIG. 5C).

The persistence of H3 Lys9 methylation at Kint2::ura4⁺ in swi6 mutant cells, but not at KΔ::ura4⁺ in swi6 mutant cells, indicates that heterochromatin formation is initiated at the cenH repeat, but requires Swi6 to spread across the entire silenced domain. These results indicate that a portion of the sequence deleted in the KΔ::ura4⁺ strain has the ability to recruit H3 Lys9 methylation by itself, and that flanking sequences present in the KΔ::ura4⁺ strain are capable of recruiting and maintaining H3 Lys9 methylation only in the presence of Swi6.

As discussed above, replacement of the cenH-containing region with ura4⁺ (KΔ::ura4⁺) results in a metastable locus that displays alternative silenced (ura4-off) and expressed (ura4-on) epigenetic states (Nakayama et al. Cell 2000;1010:307 and Grewal and Klar. Cell 1996;86:95). Once assembled, the ura4-off state is remarkably stable during both mitosis and meiosis.

Notably, the KΔ::ura4⁺ ura4-off cells exhibit considerably higher levels of Swi6 throughout their mat2/3 region when compared to ura4-on cells (Nakayama et al. Cell 2000;1010:307, and FIG. 5D). Consistent with these findings, H3 Lys9-methylation levels were higher at the mating-type region in KΔ::ura4⁺ ura4-off cells than in KΔ::ura4⁺ ura4-on cells (FIG. 5D).

Further studies examined the effect of additional copies of the swi6 and clr4 genes on association of H3 methyl-Lys9- and Swi6 with the ura4⁺ transgene sequences of KΔ::ura4⁺. For these experiments, three copies of swi6⁺ (swi6⁺-333) or six copies of clr4⁺ (clr4⁺-666) inserted at their endogenous chromosomal locations were combined with the ura4-on epiallele of KΔ::ura4⁺ through genetic crosses. Chromatin immunoprecipitation (ChIP) analysis was then performed. DNA fragments purified from whole cell extracts or DNA fragments immunoprecipitated with antibodies raised against Lys⁹-methyl histone H3 or Swi6 protein, were amplified by PCR using ura4 specific primers, that amplify both the ura4⁺ transgene replacing the cenH region (KΔ::ura4⁺), as well as the ura4 DS/E minigene located on the chromosome arm. The fold enrichment of association of Swi6 or H3 methylated-Lys9 with the KΔ::ura4⁺ sequences of the ura4-on epiallele of KΔ::ura4⁺ in the various strains, calculated relative to the whole cell extract, was determined (see Table 1).

TABLE 1
Fold enrichment values for Swi6 protein and H3 Lys9-methyl on
KΔ::ura4⁺ sequences of the ura4-on epiallele of KΔ::ura4⁺
in a wildtype strain and in strains carrying three copies of swi6⁺
(swi6⁺-333) or six copies of clr4⁺ (clr4⁺-666)
inserted at their endogenous chromosomal locations.

Fold enrichment

| S. pombe strain | Swi6 | H2 Lys9-methyl |
|---|---|---|
| wildtype | 2.6 | 1.6 |
| swi6⁺-333 | 12 | 8.5 |
| clr4⁺-666 | 3.2 | 1.5 |

This analysis showed that additional copies of swi6⁺ resulted in increases in H3 Lys9 methylation and Swi6 levels at the mat2/3 region. Notably, the observed increase in levels of Swi6 and H3 Lys9 methylation in the presence of additional copies of swi6⁺ also correlated with an increase in conversion of the ura4-on epiallele of KΔ::ura4⁺ these cells to the ura4-off epiallele. Multiple copies of clr4⁺ did not cause considerable changes in H3 Lys9 methylation and Swi6 localization.

These results underscore the importance of Swi6 in the maintenance of H3 Lys9 methylation and heterochromatin in the absence of the cenH repeat. Specifically, these results indicate that Swi6 promotes H3 Lys9 methylation at the mat locus, and that this promotion of H3 Lys9-methylation stimulates silencing of the ura4⁺ transgene at the mating locus.

The interdependence of Swi6 and H3 Lys9 methylation at the mat2/3 region suggests an “epigenetic loop” for the inheritance of the heterochromatic state, whereby H3 Lys9 methylation and Swi6 mutually support their own maintenance in a self-perpetuating manner.

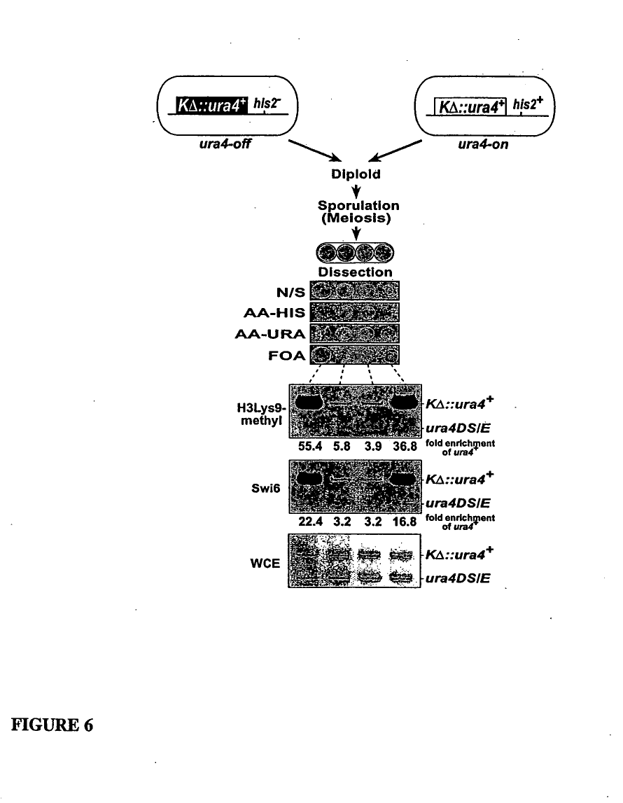

Additional studies examined whether differential localization of Swi6 and H3 Lys9 methylation patterns defining ura4-on and ura4-off epialleles of KΔ::ura4⁺ would be inherited in cis and maintained even when these epialleles are combined together into the same environment of a diploid nucleus. For these experiments, ura4-off cells and ura4-on cells of KΔ::ura4⁺0 differing in their expression of the endogenous his2 marker tightly linked to the mat locus (ura4⁺-off his2⁻ cells X ura4-on his2⁺ cells) were crossed, allowed at least 30 generation of diploid growth, sporulated, and subjected to tetrad analysis. From the tetrad, resulting haploid colonies were replicated onto non-selective medium as a positive control, medium lacking uracil (to select for ura4⁺), medium lacking histidine (to select for his2⁺), or medium containing 5-fluoroorotic acid (FOA) (to select for ura4⁻). This analysis showed a 2 Ura⁺ His⁺: 2 Ura⁻ His⁻ segregation pattern in each tetrad, indicating that the epigenetic state of the mat region is inherited in cis and segregates as a marker linked to the respective his2 alleles (FIG. 6).

Segregants from individual tetrads were also subjected to ChIP analysis using Swi6 and H3 Lys9-methyl antibodies. Consistent with the ability of heterochromatin complexes to maintain themselves, ChIP analysis of the tetrad-derived haploid ura4-off his2⁻ and ura4-on his2⁺ cells showed that the different levels of H3 Lys9 methylation and Swi6 localization corresponding to the ura4-on and ura4-off states of KΔ::ura4⁺ are inherited in cis, and are stable through meiosis (FIG. 6).

### Example 5

### cenH is a Nucleation Center for Heterochromatin and cenH-Mediated Silencing Requires the RNAi Machinery

### Materials and Methods

Yeast strains: Generation of the S. pombe strain swi6-115 (swi6⁻), in which the swi6 gene is mutated, has been described (see, e.g., Egel et al. Proc Natl Acad Sci US 1984;81:3481).

Generation of the S. pombe strain ura4::cenH-ade6⁺, in which a sequence containing the 3.6-kb cenH repeat fused to an ade6⁺ reporter gene (cenH-ade6⁺) is inserted into the ura4 gene, has been described (Ayoub et al. Genetics 2000;156:983). This strain also contains the ade6DN/N minigene at the endogenous ade6 location. Generation of the ade6DN/N has been described (Ekwall et al. Cell 1997;91:1021).

S. pombe strains carrying ura4::cenH-ade6⁺ in dcr1⁻, rdp1⁻, or ago1⁻ deletion backgrounds (see Example 1) were constructed using genetic crosses between strain ura4::cenH-ade6⁺ and the dcr1⁻, rdp1⁻, or ago1⁻ deletion strains.

Expression of ade6 sequences from ura4::cenH-ade6⁺ was scored using a colony color assay, in which yeast cells were plated on adenine-limiting YE medium and incubated at 33° C. for 3 days. The color of the colonies was then scored as red, pink, or white, where red color results from lack of ade6 expression (i.e., silencing of ade6⁺) to give an Ade⁻ phenotype, and white color indicates that ade6 is being expressed (i.e., no silencing of ade6⁺) to give an Ade⁺ phenotype.

Chromatin immunoprecipitation (ChIP) analysis: ChIP analysis was performed as described in Example 2. For these assays, S. pombe wildtype (wt), ago1⁻, dcr1⁻, and rdp1⁻ strains containing the ura4::cenH-ade6⁺ modification were used. Fragmented DNA was then purified from whole cell extracts (WCE), or immunoprecipitated with antibodies against Swi6 or histone H3 methyl-K9.

This purified whole cell extract DNA and immunoprecipitated DNA was then analyzed by PCR using primers specific for the ade6 sequences of ura4::cenH-ade6⁺ and of the ade6DN/N minigene (5′-CCG CGG TCT TCT TCC GTG CGC-3′, SEQ ID NO: 21; and 5′-GCA AGA ATG GAT CCA CCA ATC C-3′, SEQ ID NO: 22). Note that the ade6 primers amplify differently sized products from ura4::cenH-ade6⁺ and the ade6DN/N minigene. The PCR was used to incorporate a radiolabel into the products. These labeled products were then resolved on 4% polyacrylamide gels or 1% agarose gels, and visualized using a FUJI phosphoimager.

The ratios of ura4⁺ and ura4DS/E signals in the experimental (immunoprecipitated DNA) versus control (DNA from whole cell extracts) conditions were used to calculate the relative precipitated fold enrichment of associated Swi6 or histone H3 methyl-K9 proteins with the ade6⁺ sequences of cenH-ade6⁺ in the S. pombe wildtype, ago1⁻, dcr1⁻, and rdp1⁻ strains.

High resolution chromatin immunoprecipitation (ChIP) analysis: High resolution mapping of H3 Lys9-methylation and H3 Lys4-methylation was performed as previously described (Noma et al. Science 2001;293:1150). Briefly, ChIP with antibodies to H3 methylated-Lys9 or H3 methylated-Lys4 were used to measure H3 methylation levels at specific sites throughout the mat2/3 interval. For these assays, S. pombe strains that were wildtype for swi6 (swi6⁺) or that contained the swi6-115 mutation (swi6⁻), were used.

Purified whole cell extract DNA and DNA immunoprecipitated with antibodies to H3 methylated-Lys9 or H3 methylated-Lys4 was then analyzed by multiplex PCR using primers specific for a DNA fragment of the mat locus., and the actin (act1) gene (5′-CCG CGG TCT TCT TCC GTG CGC-3′, SEQ ID NO: 23; and 5′-GCA AGA ATG GAT CCA CCA ATC C-3′, SEQ ID NO: 24) as an internal amplification positive control.

The PCR was used to incorporate a radiolabel into the products. These labeled products were then resolved on 4% polyacrylamide gels and visualized and quantitated using a FUJI phosphoimager. The ratios of the mat locus and control act1 signals present in whole cell extract (WCE) DNA were used to calculate relative fold enrichment of immunoprecipitated samples. Quantitation of these results was then plotted in alignment with a map of the mat locus.

### Results and Discussion

As discussed above, the persistence of H3 Lys9 methylation at Kint2::ura4⁺ in swi6 mutant cells, but not at KΔ::ura4⁺ in swi6 mutant cells (see FIG. 5B and FIG. 5C), indicates that heterochromatin formation is initiated at the cenH repeat, but requires Swi6 to spread across the entire silenced domain. We next examined whether H3 Lys9 methylation is restricted to cenH in the absence of Swi6.

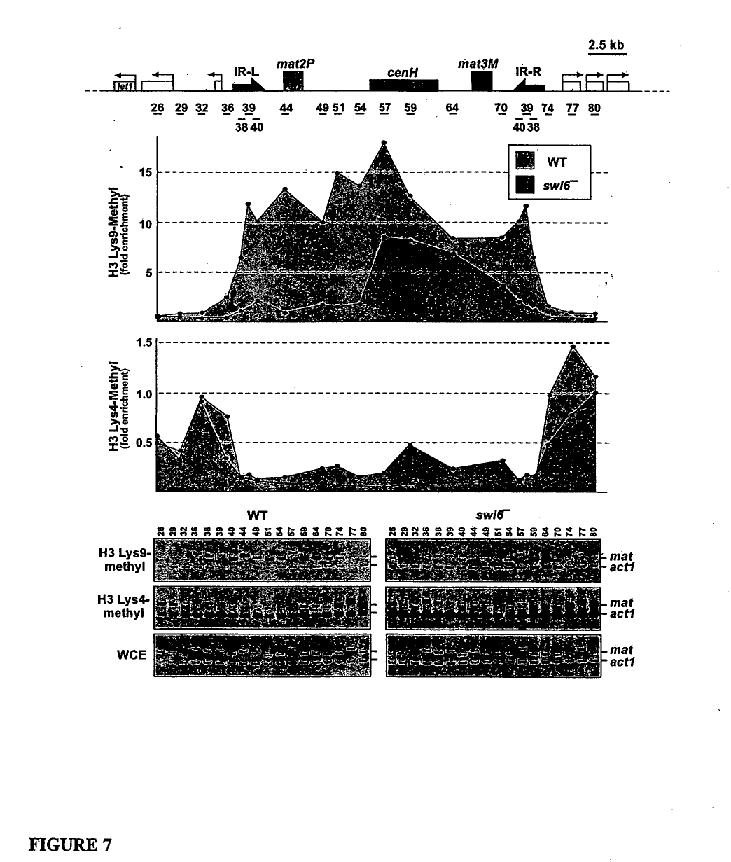

For these experiments, high resolution ChIP analysis of H3 Lys9 methylation and H3 Lys4-methylation at the mat region in wildtype and swi6 mutant strains was performed. ChIP with antibodies to methylated H3 Lys9 or H3 Lys4 were used to measure H3 methylation levels at respective sites throughout the mat2/3 interval. This analysis showed that H3 Lys9 methylation in the swi6 mutant strain was restricted to a small portion of the mat region encompassing cenH (see positions 57, 59, and 64 in FIG. 7). The loss of H3 Lys9 methylation at the mat2/3 interval in the swi6 mutant strain was correlated with only a slight increase in H3 Lys4 methylation, although a small peak directly at the transcribed portion of cenH was observed (see position 59 in FIG. 7).

These data indicate that the recruitment of H3 Lys9 methylation to the cenH repeat region occurs via a Swi6-independent mechanism, and suggest that Swi6 is required for the spreading and maintenance of H3 Lys9 methylation across the rest of the silent mat2/3 interval. These results indicate that cenH directly participates in heterochromatin formation by promoting recruitment of histone modifying enzymes and Swi6.

Further studies examined the contribution of cenH sequences to heterochromatin formation at an ectopic, otherwise euchromatic site. In these experiments was used the S. pombe strain ura4::cenH-ade6⁺, in which the 3.6-kb cenH repeat fused to an ade6⁺ reporter gene (cenH-ade6⁺) was inserted into the ura4 gene.

In wildtype cells, the cenH sequence confers repression on the reporter gene, and results in a variegated ade6 expression phenotype (Ayoub et a. Genetics 2000;156:983). These studies examined the ade6 expression phenotype in S. pombe trains carrying ura4::cenH-ade6⁺ in backgrounds containing wildtype dcr1, rdp1 and ago1 (wildtype) and in dcr1, rdp1, or ago1⁻ deletion backgrounds. The ade6 expression phenotypes of these yeast were scored using colony color assay, in which cells were plated on adenine-limiting YE medium and incubated at 33° C. for 3 days. Red or white color of the resultant colonies implies Ade⁻ and Ade⁺ phenotypes, respectively. Wildtype cells display 21% of red, 11% pink, and 68% white colonies, while dcr1⁻, rdp1⁻, or ago1⁻ RNAi machinery mutants exhibited only white colonies. Thus, deletions of ago1⁻, dcr1, or rdp1 abolish the repression of the cenH::ade6⁺ reporter.

ChIP analysis was then performed to determine if formation of heterochromatin on the ade6⁺ sequences of cenH-ade6⁺, like silencing of cenH-ade6⁺, is dependent upon the RNAi machinery. In this analysis, the levels of Swi6 and H3 Lys9 methylation were determined at sequences of cenH::ade6⁺ in dcr1⁻, rdp1⁻, or ago1⁻ RNAi machinery mutant strains and a wildtype strain. DNA fragments purified from whole cell extracts or DNA fragments immunoprecipitated with antibodies raised against Swi6 or H3 Lys⁹-methyl histone H3 were amplified by PCR using primers specific for the ade6 sequences of ura4::cenH-ade6⁺ and of the ade6DN/N minigene. The fold enrichment of association of Swi6 or H3 methylated-Lys9 with the ade6⁺ sequences of cenH-ade6⁺ in the various strains, calculated relative to the whole cell extract was determined (see Table 2).

TABLE 2
Fold enrichment values for Swi6 protein and H3 Lys9-methyl on
cenH-ade6⁺ in dcr1⁻, rdp1⁻, or ago1⁻ RNAi machinery
mutant strains and in a wildtype strain.

Fold enrichment

| S. pombe strain | Swi6 | H2 Lys9-methyl |
|---|---|---|
| wildtype | 11.6 | 50.9 |
| ago1⁻ | 1.2 | 0.9 |
| dcr1⁻ | 1.2 | 1.2 |
| rdp1⁻ | 1.0 | 0.8 |

These data show that cenH-mediated heterochromatin formation at an ectopic site requires the RNAi machinery. Thus, in yeast with intact RNAi machinery (wildtype), the observed silencing of ade6⁺ at the ectopic site, correlates with preferential enrichment of both H3 Lys9 methylation and Swi6. Conversely, in yeast with RNAi machinery defects (dcr1⁻, rdp1⁻, or ago1⁻ deletion strains), the lack of ade6⁺ silencing at the ectopic site, correlates with a lack of H3 Lys9-methylation and Swi6. These data demonstrate that the cenH repeat is sufficient to induce heterochromatin formation, and that this formation requires the RNAi machinery.

Overall, these date show that deletions of ago1, dcr1, or rdp1 abolish the repression of the cenH::ade6⁺ reporter. Furthermore, in these mutants, the cenH::ade6⁺ sequence was no longer able to recruit and/or maintain H3 Lysine 9 methylation and Swi6. These data indicate that cenH-induced silencing and the corresponding H3 Lys9 methylation and Swi6 localization to the ectopic domain require the RNAi machinery.

### Example 6

### The RNAi Machinery is Required for Initiation of Heterochromatin Assembly

### Materials and Methods

Yeast strains: S. pombe strains carrying the Kint2::ura4+ integration in dcr1−, rdp1−, or ago1− deletion backgrounds were constructed using genetic crosses between strain Kint2::ura4⁺, in which the ura4⁺ transgene is inserted at the cenH region between mat2P and mat3M of the mating type region (see Example 4), and the dcr1−, rdp1−, or ago1− deletion strains (see Example 1).

In experiments where the epigenetic imprint governing silencing at the mat region was erased by treatment with the deacetylase inhibitor Trichostatin A (TSA), S. pombe trains carrying the Kint2::ura4⁺ integration in dcr1−, rdp1−, or ago1− deletion backgrounds were treated with 35 μg/ml TSA for ten generations, and were allowed to grow for an additional ten generations in the absence of TSA.

The generation of an S. pombe strain carrying a deletion of clr4 (clr4Δ), the Kint2::ura4+ reporter gene, and the ura4DS/E minigene was described previously (Nakayama et al. Science 2001;292: 110).

The S. pombe clr4Δ strain carrying the Kint2::ura4⁺ reporter gene and ura4DS/E minigene was crossed with the dcr1−, rdp1−, or ago1− deletion strains (see Example 1) to construct diploid strains that are heterozygous for clr4A and homozygous for ago1Δ, dcr1Δ or rdp1Δ (e.g. clr4+/clr4Δ dcr1−/dcr1−).

Iodine staining assessment of mating-type switching: The efficiency of mating-type switching was assessed by iodine staining as described in Example 1.

Tetrad analysis: Mating, sporulation and tetrad analysis of S. pombe strains was performed as previously described (see, for example, Moreno et al. Methods Enzymol 1991;194:795).

Chromatin immunoprecipitation (ChIP) analysis: ChIP analysis was performed as described in Example 2. For these assays, S. pombe wildtype (WT), ago1−, dcr1−, and rdp1− strains containing the Kint2::ura4+ modification were used. Fragmented DNA was purified from whole cell extracts, or immunoprecipitated with the antibodies against Swi6 or histone H3 methyl-Lys9. This purified whole cell extract DNA and immunoprecipitated DNA was then analyzed by PCR using primers specific for the ura4 sequences of the ura4+ transgene of Kint2::ura4+ and ura4-DS/E minigene (See Example 2). Note that this is a competitive PCR strategy, whereby one primer-pair amplifies different size PCR products from the full-length ura4+ located at Kint2::ura4+ and from the control ura4DS/E minigene. The PCR was used to incorporate a radiolabel into the products. These labeled products were then resolved on 4% polyacrylamide gels and visualized and quantitated using a FUJI phosphoimager.

The ratios of ura4⁺ and ura4DS/E signals in the experimental (immunoprecipitated DNA) versus control (DNA from whole cell extracts) conditions were used to calculate the relative precipitated fold enrichment of associated Swi6 or histone H3 methyl-K9 proteins in strains that were wildtype for swi6 (swi6+) or that contained the swi6-115 mutation (swi6-).

### Results and Discussion

To investigate the role of the RNAi machinery in heterochromatin assembly at the endogenous mat region, the Kint2::ura4⁺ reporter was introduced into the mat region of the respective RNAi mutant backgrounds using genetic crosses.

Serial dilution plating assays in the presence and absence of 5-fluoroorotic acid (FOA) were performed to measure Kint2::ura4⁺ expression in the dcr1⁻, rdp1⁻, or ago1⁻ deletion strains. In this assay, silencing of the ura4⁺ transgene enables the yeast to survive in media containing FOA. Surprisingly, the survival of the dcr1⁻, rdp1⁻, or ago1⁻ deletion strains of Kint2::ura4⁺ was indistinguishable from the wildtype, indicating that silencing of ura4 at the mat locus was intact in the RNAi machinery mutant strains. In addition, the efficiency of mating-type interconversion as determined by iodine staining, which depends upon the heterochromatic structure at the mat2/3 region, was unaffected in the RNAi mutant strains.

ChIP analysis was next performed to determine if maintenance of heterochromatin on the ura4 sequences of Kint2::ura4⁺, like silencing of ura4 at the mat locus, is intact in the RNAi machinery mutant strains. This analysis was performed on S. pombe wildtype, ago1⁻, dcr1⁻, and rdp1⁻ strains containing the Kint2::ura4⁺ modification. DNA fragments purified from whole cell extracts or DNA fragments immunoprecipitated with antibodies raised against Swi6 or H3 Lys⁹-methyl histone H3 were amplified by PCR using primers using ura4 specific primers, that amplify both the ura4⁺ transgene of Kint2::ura4⁺, as well as the ura4 DS/E minigene located on the chromosome arm. The fold enrichment of association of Swi6 or H3 methylated-Lys9 with the Kint2::ura4⁺ sequences of the Kint2::ura4⁺-containing mat region, calculated relative to the whole cell extract, was determined (see Table 3).

TABLE 3
Fold enrichment values for Swi6 protein and H3 Lys9-methyl
Kint2::ura4⁺ sequences of the Kint2::ura4⁺-containing
mat region in dcr1⁻, rdp1⁻, or ago1⁻ RNAi machinery
mutant strains and in a wildtype strain.

| | Fold enrichment | Fold enrichment |
|---|---|---|
| S. pombe strain | Swi6 | H2 Lys9-methyl |
| wildtype | 16.9 | 31.2 |
| ago1⁻ | 12.8 | 35.8 |
| dcr1⁻ | 16.0 | 36.3 |
| rdp1⁻ | 19.5 | 39.0 |

This data shows that the levels of Swi6 protein and H3 Lys9 methylation of ura4 sequences of Kint2::ura4⁺ at the mat2/3 region are comparable in wildtype and dcr1⁻, rdp1⁻, or ago1⁻ deletion strains. These results indicate that the RNAi machinery is dispensable for the maintenance of a preassembled heterochromatic state.

The role of RNAi components in the establishment of silencing was addressed by examining their involvement in the initiation step of heterochromatin formation. The deacetylase inhibitor Trichostatin A (TSA) has previously been shown to erase the epigenetic imprint governing silencing at the mat region (Grewal et al. Genetics 1998; 150:563).

The epigenetic imprint governing silencing at the mat region in S. pombe trains carrying the Kint2::ura4⁺ integration in dcr1⁻, rdp1⁻, or ago1⁻ deletion backgrounds was erased by treatment with TSA, and then the cells were allowed to recover to 10 generation. Then expression of the ura4 transgene from Kint2::ura4⁺ was assessed by serial dilution analysis (top). In this assay, silencing of the ura4⁺ transgene enables the yeast to survive in media containing FOA. The survival of the dcr1⁻, rdp1⁻, or ago1⁻ deletion strains of Kint2::ura4⁺ was markedly reduced compared to the wildtype, indicating that following TSA-treatment silencing of the ura4 transgene was not re-established in these cells.

It was next determined if the establishment of heterochromatin on the ura4 sequences of Kint2::ura4⁺, like silencing of ura4 at the mat locus, is disrupted in the RNAi machinery mutant strains. For this experiment, ChIP analysis was performed to determine the levels of Swi6 and H3 Lys9 methylation at Kint2::ura4⁺ after recovery from TSA treatment. This analysis was performed on S. pombe wildtype, ago1⁻, dcr1, and rdp1 strains containing the Kint2::ura4+modification, which had been treated with TSA for ten generations, and then recovered for ten generations in the absence of TSA. DNA fragments purified from whole cell extracts or DNA fragments immunoprecipitated with antibodies raised against Swi6 or H3 Lys⁹-methyl histone H3 were amplified by PCR using primers using ura4 specific primers, that amplify both the ura4⁺ transgene of Kint2::ura4⁺, as well as the ura4 DS/E minigene located on the chromosome arm. The fold enrichment of association of Swi6 or H3 methylated-Lys9 with the Kint2::ura4⁺ sequences of the Kint2::ura4⁺-containing mat region, calculated relative to the whole cell extract, was determined (see Table 4).

TABLE 4
Fold enrichment values for Swi6 protein and H3 Lys9-methyl
Kint2::ura4⁺ sequences of the Kint2::ura4⁺-containing mat
region in dcr1⁻, rdp1⁻, or ago1⁻ RNAi machinery mutant
strains and in a wildtype strain following TSA treatment and recovery.

Fold enrichment

| S. pombe strain | Swi6 | H2 Lys9-methyl |
|---|---|---|
| wildtype | 12.5 | 113.5 |
| ago1⁻ | 1.9 | 8.5 |
| dcr1⁻ | 1.8 | 3.7 |
| rdp1⁻ | 2.0 | 4.9 |

These data demonstrate that after recovery from TSA treatment, the levels of H3 Lys9 methylation and Swi6 associated with ura4 sequences of Kint2::ura4⁺ were considerably higher at the mat region of wildtype cells when compared to RNAi mutant cells. This result indicates that the RNAi deletion strains are unable to efficiently reestablish the heterochromatic state after it has been erased. In other words, RNAi mutants are defective in the establishment of heterochromatin.

Thus, after treatment with TSA and an additional 10 generations of growth in the absence of TSA, wildtype cells fully reestablished silencing and reestablished heterochromatin formation at the mat2/3 locus. In a striking contrast, ago 1⁻, dcr1⁻ and rdp1⁻ strains were defective in the establishment of heterochromatin at the mat2/3 locus, and only a relatively small proportion of cells acquired silencing of Kint2::ura4⁺.

To genetically test the role of RNAi machinery in the initiation of heterochromatin formation, a clr4A strain carrying the Kint2::ura4⁺ reporter gene was used to construct diploid strains heterozygous for clr4A and homozygous for ago1⁻, dcr1⁻ or rdp1⁻ (e.g. clr4⁺/clr4Δ dcr1⁻/dcr1⁻). Since Clr4 is the sole H3 Lys9-specific methyltransferase in fission yeast, the mat locus propagated in a clr4A background is completely devoid of H3 Lys9 methylation and Swi6 protein (Nakayama et al. Science 2001;292:110).

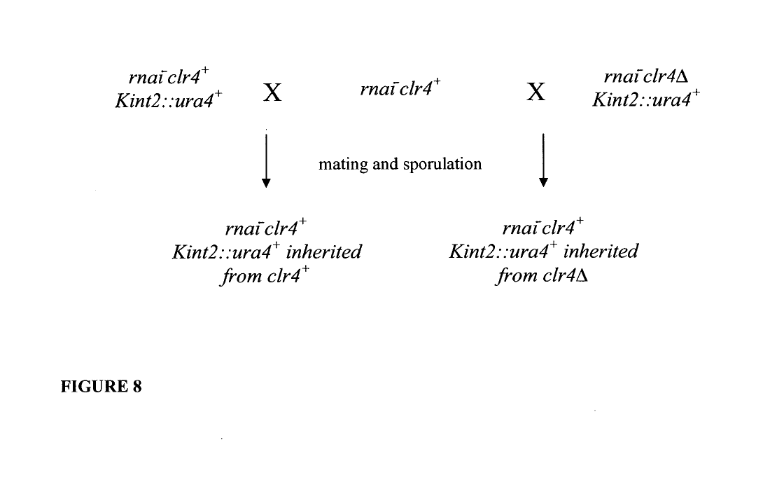

The Kint2::ura4⁺-containing mating type region derived from either a wildtype or clr4Δ background was introduced into the various RNAi mutant backgrounds by genetic crosses (FIG. 8). Diploids were sporulated to obtain ago1⁻ clr4⁺, dcr1⁻ clr4⁺ or rdp1⁻ clr4⁺ haploid segregants. Phenotypic analysis of the these segregants that contain a functional H3 Lys9 methyltransferase but lack RNAi machinery revealed severe defects in heterochromatin assembly at the mat locus, as observed by both Kint2::ura4⁺ expression and the decreased efficiency of mating-type interconversion.

To assay mating-type switching, which depends on heterochromatin assembly at the mat locus, colonies were replicated onto sporulation medium (PMA⁺) and stained with iodine vapors. Dark staining indicates efficient mat switching, while light or sectored staining indicates defects in switching and heterochromatin formation. This analysis indicated that where the Kint2::ura4⁺-containing mating type region was derived from a wildtype clr4 background, mating type switching was efficient in the RNAi mutant strains. Conversely, where the Kint2::ura4⁺-containing mating type region was derived from a clr4A background, no mating type switching was observed in the RNAi mutant strains.

ChIP analysis was next performed to determine the levels of Swi6 and H3 Lys9 methylation at Kint2::ura4⁺ sequences at the mat region in RNAi machinery mutants containing a Kint2::ura4⁺-containing mat region derived from clr4A or wildtype clr4 backgrounds. For this experiment, DNA fragments purified from whole cell extracts or DNA fragments immunoprecipitated with antibodies raised against Swi6 or H3 Lys⁹-methyl histone H3 were amplified by PCR using primers using ura4 specific primers, that amplify both the ura4⁺ transgene of Kint2::ura4⁺, as well as the ura4 DS/E minigene located on the chromosome arm. The fold enrichment of Swi6 or H3 Lys⁹-methyl histone at the Kint2::ura4⁺-containing mat region derived from clr4Δ versus wildtype clr4 backgrounds, calculated relative to the whole cell extract control, was determined (see Table 5).

TABLE 5
Fold enrichment values for Swi6 protein and H3 Lys9-methyl on a
Kint2::ura4⁺-containing mat region derived from clr4Δ or
wildtype clr4 backgrounds in various RNAi mutant strains.

| | | |
|---|---|---|
| Kint2::ura4⁺ | RNAi machinery | Fold enrichment |

| inherited from: | mutant | Swi6 | H2 Lys9-methyl |
|---|---|---|---|
| clr4+ | ago1⁻ | 13.0 | 61.8 |
| | dcr1⁻ | 14.1 | 63.4 |
| | rdp1⁻ | 14.0 | 51.4 |
| clr4Δ | ago1⁻ | 5.1 | 15.6 |
| | dcr1⁻ | 3.6 | 10.5 |
| | rdp1⁻ | 1.9 | 2.9 |

This analysis showed that the RNAi mutant strains with a Kint2::ura4⁺-containing mat region derived from the clr4A background have substantially less H3 Lys9 methylation and Swi6 protein associated with the Kint2::ura4⁺ sequences than strains in which the Kint2::ura4+-containing mat region was derived from a wildtype clr4 background.

The results of these mating-type switching and ChIP analysis studies show that RNAi mutants cannot efficiently initiate heterochromatin formation. In other words, the RNAi machinery is required for the initiation of heterochromatin.

It is noteworthy, however, that after introduction of the clr4⁺ allele the proportion of cells with a silenced mat region increases each generation in an inefficient and highly stochastic manner, indicating that heterochromatin formation eventually does occur in the absence of the RNAi machinery, likely via an alternative Swi6-based mechanisms.

These data indicate that while the RNAi mutant strains are able to maintain a silenced mat region when it is derived from a wildtype parent, they are unable to effectively establish silencing at a mat region rendered epigenetically active by TSA treatment or propagation in a clr4Δ background.

These findings are consistent with the inefficient establishment of silencing observed in KΔ::ura4⁺ cells carrying deletion of cenH (Grewal and Klar. Genetics 1997;146:1221), and further suggest that the RNAi machinery and the cenH repeat operate in the same pathway to establish heterochromatin.

These discoveries define sequential events in the assembly of heterochromatin at the mating-type region of fission yeast and show that the establishment of epigenetic silencing requires an initial nucleation event, and is distinct in this respect from mechanisms that act in cis to reinforce propagation of the heterochromatic state. Without intending to be limited by mechanism, these results are consistent with the following model: transcripts derived from cenH are processed by the RNAi machinery, and resulting RNA intermediates directly recruit HDAC and H3 Lys9 methyltransferase activities to the mat locus. This initial recruitment nucleates heterochromatin by creating H3 Lys9 methylated binding sites for the Swi6 protein. Once bound to chromatin, Swi6 serves as a platform for the recruitment of histone modifying activities that create additional Swi6 binding sites on adjacent nucleosomes, thus enabling spreading to occur in a stepwise manner. Upon chromosome replication, parental histone H3 and Swi6 are hypothesized to segregate randomly to the daughter chromatids, and Swi6-based activities serve to imprint the parental histone modification pattern onto newly assembled nucleosomes by the same mechanism in which they promote spreading in cis.

The mechanism proposed above is reminiscent of mammalian X-chromosome inactivation, where an H3 Lys9 methylation hotspot upstream of the Xist locus serves to initiate the cooperative spreading of Xist non-coding RNA and H3 Lys9 methylation across the entire inactive X (Heard et al. Cell 2001;107:727). Significantly, once silencing is established, Xist RNA becomes dispensable and the heterochromatic state persists in the absence of the initial stimulus (Cohen and Lee. Curr Opin Genet Dev 2002;12:219). It is considered, therefore, that mechanisms involving RNAi-like processes may also operate in the lineage-specific establishment of silenced chromatin domains during development.

It is remarkable that, in fission yeast, the mating-type locus utilizes a repetitive element to organize a highly specialized chromatin structure that controls transcriptional silencing, recombinational suppression, and the non-random utilization of silent cassettes during mating-type switching. Similar processes may influence a variety of chromosomal functions important to preserve genomic integrity, such as prohibition of wasteful transcription and suppression of deleterious recombination between repetitive elements. In this regard, it should be noted that the presence of large, repetitive heterochromatic regions is widespread among eukaryotes, and in Drosophila, plants, mammals, and some fungi, the introduction of repetitive sequences of diverse origin can stimulate pathways leading to heterochromatin formation (Birchler et al. Curr Opin Genet Dev 2000; 10:211; Hsieh and Fire. Annu Rev Genet 2000;34:187; Selker. Cell 1999;97:157; and Pal-Bhadra et al. Mol Cell 2002;9:315).

### Example 7

### RNAi and DNA Methylation

As discussed above, in S. pombe, heterochromatin is marked by methylation of histone H3 at lysine 9, while analogously in mammals, heterochromatin is marked by methylation of histone H3 at the lysine 9 and/or lysine 27 residue. Thus, the results reported for the effects of RNAi on heterochromatin and histone methylation in S. pombe cells constitute useful analogs by which to understand the effects of RNAi on heterochromatin and histone methylation in mammalian cells.

Unlike S. pombe, filamentous fungi such as Ascobolus immersus and Neurospora crassa have DNA methylation as well as histone modification. In Neurospora crassa, cytosine methylation of ribosomal DNA and relics of RIP (repeat induced point mutation) were found to require the histone H3 lysine 9 methyltransferase dim-5 (Tamaru and Selker. Nature 2001;414:277). Homology-dependent silencing (also known as “quelling”) is also often associated with DNA methylation in Neurospora, but it is unaffected in dim-2 DNA methyltransferase mutants (Cogoni et al. EMBO J. 1996;15:3153). Instead, genes encoding components of the RNAi machinery are required (Cogoni and Macino Nature 1999;399:166 and Shiu et al. Cell 2001;107:905). Our results are consistent with these observations, and suggest that histone H3 lysine-9 methylation is the direct consequence of RNAi, while DNA methylation is an indirect consequence of RNAi.

In the mouse, X-inactivation and Igf2r imprinting are mediated in cis by specific non-coding antisense RNA (Avner and Heard. Nat Rev Genet 2001; 2:59 and Sleutels et al. Nature 2002;415:810). At least in the case of X inactivation, histone H3 lysine 9 methylation immediately follows the appearance of the non-coding Xist transcript, which is regulated by an antisense RNA, Tsix and by promoter methylation (Heard et al. Cell 2001;107:727). This configuration of transcripts and chromatin changes parallels the arrangement we presently describe for S. pombe.

In the case of both X-inactivation and genomic imprinting, the silenced sequences are commonly associated with characteristic patterns of DNA methylation, whereas the corresponding unsilenced sequences on the cognate chromosome of the diploid pair are not associated with such patterns of DNA methylation.

The Prader-Willi syndrome imprinting center (on human chromosome 15 and mouse chromosome 7) shows both methylation of CpG DNA sequences and Histone H3 Lys-9 methylation of the silenced maternal chromosome. Furthermore, in mouse embryonic stem cells, maintenance of CpG methylation at the Prader-Willi syndrome imprinting center requires the function of the G9a histone H3 Lys-9/Lys-27 methyltransferase (Xing et al. J Biol Chem 2003;278:14996-15000).

Therefore, the results presented herein suggest that in mammals sequence-specific histone modification is targeted by the RNA interference machinery during X inactivation and in genomic imprinting, which in turn leads to DNA methylation and epigenetic silencing. More generally, the results provide a link between RNA interference and DNA methylation. Without intending to be limited by mechanism, the applicants propose that dsRNA derived from repeated sequences triggers RNAi, which initiates histone H3 lysine-9 methylation. Histone modification then signals DNA methylation. This mechanism could guide eukaryotic DNA methyltransferases to specific regions of the genome, such as transposable elements, even though they have little sequence specificity in themselves. Such an arrangement could be reinforced by maintenance methyltransferase activity, as well as by the deacetylation of histones guided by methyl DNA binding complexes.

### Example 8

### RNAi Machinery is Required for the Fidelity of Chromosome Segregation

### Materials and Methods

Yeast strains: The S. pombe dcr1⁻, rdp1⁻, and ago1⁻ deletion strains were generated as in Example 1. Standard conditions were used for growth, sporulation, tetrad analysis, and construction of diploids. (see, for example, Moreno et al. Methods Enzymol 1991;194:795).

Immunofluorescence (IF) analysis: IF was carried out as previously described (Nakayama, et al. EMBO J. 2001;20:2857).

Microscopic analysis: Samples were analyzed with a Zeiss Axioplan2 fluorescent microscope. For deconvolution, images were collected at 0.2-μm intervals along the z axis and subjected to volume deconvolution using the nearest three neighbors method. OPENLAB software (Improvision) was used for all analyses.

### Results and Discussion

Given that heterochromatin formation has been linked to centromere function in fission yeast (Allshire et al. Genes Dev 1995;9:218), and in other systems (Murphy and Karpen. Cell 1995;82:599; Kellum & Alberts. J Cell Sci 1995;108:1419; Peters et al. (2001) Cell 107, 323-337), and that abnormal segregation patterns are often observed in crosses involving the RNAi mutant strains, several experiments were performed to investigate whether mitotic chromosome segregation was disrupted in the RNAi mutants.

Serial dilution analysis of cell growth revealed that the RNAi mutant yeast strains (ago1⁻, dcr1⁻ and rdp1⁻) showed greater sensitivity to the microtubule destabilizing drug thiabendazole than wild-type cells. These findings indicated that the process of chromosome segregation is not robust in the mutant strains. The rates of chromosome loss for wild-type and mutant strains were measured by the analysis of homozygous diploids. The data, presented in Table 6, revealed that ago1⁻, dcr1⁻, and rdp1⁻ diploids have significantly higher rates of nondisjunction (45- to 60-fold) than their wild-type counterparts. Chromosome segregation was dependent on the RNAi machinery.

TABLE 6
Rates of chromosome loss for wild-type and mutant strains as measured
by the breakdown of homozygous diploids.

| | Chromosome Loss Rate | | |
|---|---|---|---|
| S. pombe strain | Per Division | Fold Increase | N |
| WT | 4.6 × 10⁻⁴ | | 4343 |
| ago1⁻ | 2.6 × 10⁻² | 56.5 | 2034 |
| dcr1⁻ | 3.1 × 10⁻² | 67.4 | 2038 |
| rpd1⁻ | 2.1 × 10⁻² | 45.7 | 2307 |

Immunofluorescence studies were then carried out with either anti-tubulin antibodies to visualize microtubules or with the stain 4′,6-diamidino-2-phenylindole (DAPI) to visualize chromosomes. RNAi mutant cells revealed a number of aberrant features, including lagging chromosomes in late anaphase mutant cells with a fully elongated spindle, highly elongated cells containing multiple and/or fragmented nuclei, and late mitotic cells in which the majority of DNA was segregated to one of the two daughter nuclei. These results demonstrate that the ago1, dcr1, and rdp1 gene products play an important role in the proper segregation of chromosomes through mitosis.

### Example 9

### Centromeric Cohesion is Defective in RNAi Mutants

### Materials and Methods

Yeast strains, Cell culture, Immunofluorescence analysis, and Microscopic imaging: These have already been described in the previous Examples.

Fluorescent in Situ Hybridization (FISH): FISH was performed as described (Matsuura et al. Genetics 1999;152:1501). Briefly, log-phase cells were fixed in 3% paraformaldehyde for 2 min at room temperature followed by the addition of 25% glutaraldehyde to a final concentration of 0.2%, and incubated at 26° C. for 1 h. After fixation, cells were washed sequentially, treated with 10 μg/ml RNase A for 2 h, and hybridized overnight with the Cy3-labeled 15-kb insert spanning the dg and dh centromeric repeats, contained on the pRS 140 plasmid (Chikashige et al. Cell 1989;57:739).

Chromatin immunoprecipitation (ChIP): ChIPs were performed as described (Nakayama et al. EMBO J. 2001; 20:2857). DNA recovered from immunoprecipitated chromatin fractions or whole cell crude extracts was subjected to multiplex PCR analysis using the dh383 primer pair (Nakagawa et al. Genes Dev 2002;16:1766) recognizing the dh centromeric repeat and the control ade6DN/N primer pair fragment from ade6⁺ gene. Fold enrichment was calculated by taking the ratio of intensities of the dh band and the ade6⁺ band from the ChIP fraction, and dividing that by the ratio of their intensities in the whole cell crude extracts.

### Results and Discussion

In fission yeast, the preferential recruitment of the cohesin complex at centromeres depends on the presence of heterochromatin, as Swi6 directly recruits cohesin to the outer repeats (Bernard et al. Science 2001;294:2539; Nonaka et al. Nat Cell Biol 2001;4:89). The studies here examined centromeric cohesion by analyzing a haploid strain that expresses a LacI-GFP fusion protein, and contains the LacO DNA repeat inserted at the lys1 locus linked to centromere 1 (cen1-GFP) (Nabeshima et al. Mol Biol Cell 1998;9:3211). Because fission yeast cells spend most of the cell cycle in the G2 phase, the single GFP spot commonly observed in wild-type cells corresponds to the sister chromatids of chromosome I joined at the centromere. Immunofluorescent studies revealed two GFP foci in a small percentage of wildtype cells (10.8%), but in a significantly higher percentage of RNAi mutant cells (28.7%, 42.7%, and 35.7% in ago1⁻, dcr1⁻, and rpd1⁻ strains, respectively). In the mutant cells, the two cen1-GFP foci were often in the general vicinity of each other, likely indicating that sister chromatid cohesion is not disrupted along the entire length of the chromosome. This finding is consistent with independent mechanisms regulating the recruitment of cohesin to chromosome arms and centromeres (Bernard et al. Science 2001;294:2539; Nonaka et al. Nat Cell Biol 2001;4:89).

In a further analysis of centromere localization in wild-type and RNAi mutant cells, FISH was carried out with a 15-kb probe that hybridizes to the outer repeats of all three centromeres. The number of spots was counted by microscopic inspection of >100 cells for each strain. Consistent with the analysis using cen1-GFP reporter, a significantly higher percentage of cells with two spots was observed in RNAi mutant cells (49.5%, 42.6%, and 42.2% in the ago1⁻, dcr1, and rdp1 strains, respectively) than in wild-type cells (19.4%).

In a direct examination of the concentration of cohesin at centromeres, ChIp analysis was carried out in wild-type and mutant strains, each encoding an epitope-tagged version of the cohesin subunit Rad21. Although Rad21 showed a pronounced enrichment in the centromeres of wild-type cells (3.2-fold), it showed little or no enrichment in the centromeres of RNAi mutant cells (0.83-, 0.49-, and 1.59-fold in the ago1⁻, dcr1⁻, and rpd1⁻ strains, respectively). This defect in centromere cohesion is most likely a product of the observed defects in Swi6 localization to centromeres in the RNAi mutant strains (See Example 4 and Table 1). This finding suggests that one possible cause for aberrant segregation is defective recruitment of cohesin to centromeres.

### Example 10

### RNAi Machinery is Required for Telomere Clustering but is Dispensable for Telomere Maintenance

### Materials and Methods

Yeast strains, Cell culture, Immunofluorescence analysis, and Microscopic imaging: These have already been described in the previous Examples.

Southern blot analysis: Genomic DNA was digested with EcoRI, separated by agarose gel electrophoresis, and transferred to a nitrocellulose membrane by standard procedures. The blot was probed with an α-³²P-labeled 300-bp fragment derived from the terminal telomeric repeat contained on the pAMP002 plasmid (Kanoh and Ishikawa. Curr Biol 2001; 11: 1624).

### Results and Discussion

Recent studies have implicated RNA in the formation of higher-order chromosomal structures (Maison et al. Nat Genet 2002;30:329). In fission yeast, heterochromatin is found at the centromeres, telomeres, and mating-type region, and can be visualized by immunostaining for Swi6. Previous studies have shown two to five discrete Swi6 foci in interphase cells, of which approximately one is a cluster of all three centromeres. Telomeres form two to four foci of Swi6, with two being most common, and a faint spot corresponds to the mating-type region (Ekwall et al. J Cell Sci 1996;109:2637). In some mutants defective in centromeric silencing, such as clr4 and rik1, Swi6 becomes entirely delocalized from the chromosomes and is present in a diffuse pattern throughout the nucleus andnucleolus (Ekwall et al. J Cell Sci 1996;109:2637).

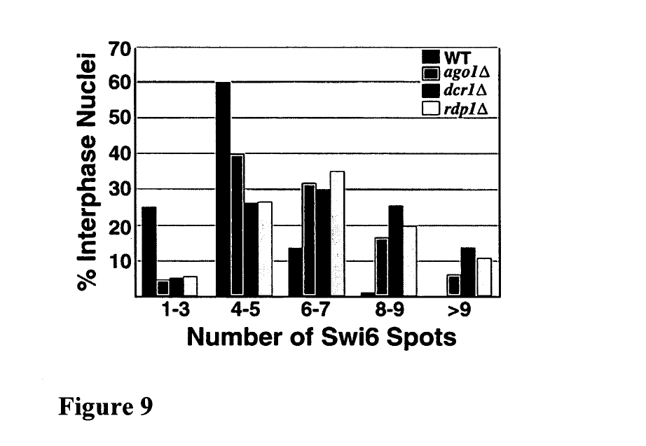

Given that the heterochromatic regions of fission yeast cluster together into higher order structures reminiscent of the pericentric heterochromatin of higher eukaryotes, the effects of deletion of the RNAi machinery on Swi6 localization was examined by using immunofluorescence. Surprisingly, most interphase mutant cells had a greater number of Swi6 foci than wild-type cells, though these foci were generally smaller in size and less intense (FIG. 9). Given that the RNAi mutants are defective in Swi6 localization at centromeres (See Example 4 and Table 1), additional experiments addressed whether the additional foci were a result of defective telomere clustering. Taz1 is a protein that binds exclusively to telomere repeats, and is commonly used as a marker for their localization (Cooper et al. Nature 1997; 385:744). Coimmunofluorescence experiments with Swi6 and Taz1 revealed that most of the Swi6 foci observed in interphase mutant cells colocalized with Taz1, indicating that the mitotic clustering of telomeres is defective in the mutant strains. However, the localization of telomeres to the nuclear periphery did not appear to be affected.

How the RNAi machinery affects mitotic telomere clustering is not clear. RNA intermediates produced by RNAi may promote the chromosomal association of telomeres by acting as a “glue” to hold distinct heterochromatic regions from dispersed genomic locations into a common structure. In this regard, it should be noted that the formation of higher-order heterochromatic structures in eukaryotes with more complex genomes requires that loci from multiple chromosomal regions cluster together. In fission yeast, similar processes may operate in the clustering of telomeres.

It is interesting that, in contrast to telomeres, no defects in the clustering of nonhomologous centromeres were observed. This is consistent with previous studies showing that the fission yeast centromeres are divided into two distinct domains, and that the factors that interact with these domains might contribute to redundant mechanisms responsible for centromeric clustering (Partridge et al. Genes Dev 2000;14:783). Although the RNAi machinery is required for localization of Swi6 and at the highly repetitive outer centromeric region, it is dispensable for silencing at the central core, which is the site of kinetochore formation and occupied by entirely different factors such as Mis6 and Cnp1 (Saitoh et al. Cell 1997;90: 131; Takahashi et al. Science 2000;288:2215).

The role of RNAi in telomere maintenance was examined by analyzing the effects of RNAi mutations on silencing of a his3⁺ reporter gene inserted within a telomere of chromosome 1 (tell::his3⁺) (Nimmo et al. Nature 1998;392:825). Serial dilution analyses of the strains containing the tell::his3⁺ reporter revealed that maintenance of telomeric silencing is not affected in the RNAi mutant background. In addition, an examination of telomeric DNA by Southern blot analysis revealed no difference in the length of telomeric repeats in wild-type and mutant cultures, indicating that the pathways that maintain the length of telomeres are intact. The lack of silencing defects in the RNAi mutant strains probably reflects the ability of telomeric repeat DNA to directly recruit telomere-specific silencing factors.

In light of the above results showing that telomere maintenance does not appear to be affected in RNAi mutants, the precise role of mitotic telomeric clustering remains unclear. It is possible that clustering facilitates the establishment of heterochromatin by concentrating telomere ends in specialized nuclear compartments that are enriched in silencing factors. Another possibility is that mitotic telomere clustering facilitates the transition to meiosis, where the clustering of telomeres at the spindle pole body during prophase helps align homologous chromosomes (Niwa et al. EMBO J. 2000;19:3831; Scherthan et al. J Cell Biol 1994;127:273).

### Example 11

### Meiotic Chromosome Segregation and Meiotic Telomere Clustering are Disrupted in RNAi Mutants

### Materials and Methods

Yeast strains, Cell culture, Immunofluorescence analysis, and Microscopic imaging: These have been described in the previous Examples. For meiotic IF, mating-type switching-competent (h⁹⁰) mid-log phase cells were concentrated by centrifugation, spotted on solid pombe minimal medium (PMA⁺), and cultured for 15-20 h at 26° C. for sporulation.

### Results and Discussion

Chromosome segregation during meiosis and mitosis are distinct processes. At the first reductional meiotic division, sister chromatids remain associated at their centromeres and move together to the same pole. During the second meiotic division, sister chromatids separate from each other and segregate to opposite poles. This process requires tight regulation of kinetochore orientation and meiotic centromere cohesion (Watanabe and Nurse. Nature 1999;400:461).

In a study of meiotic chromosome segregation, strains carrying the cen1-GFP marker described in Example 10 were sporulated and examined by miscroscopy to follow the segregation of the cen1 locus in live tetrads. A normal meiosis results in an ascus in which each of the four spores contain a single cen1-GFP spot. Missegregation of cen1 will result in a tetrad in which one spore contains two cen1-GFP spots, and another spore lacks a GFP spot.

Because fission yeast undergo ordered meiosis (the two adjacent spores at each end of the tetrad are products of the same second meiotic division), it can be deduced whether a given missegregation event occurred during the first or second meiotic division. Such an analysis with the RNAi mutants revealed four phenotypic classes. Class I represents normal meiotic segregation, where each spore receives one copy of chromosome I. Class II represents a missegregation event during one of the two second meiotic divisions. Class III is caused by missegregation during both of the second meiotic divisions. Class IV is caused by missegregation of a single cen1-GFP chromatid during the first meiotic division. The frequency of each class for each strain is listed in Table 7, which reveals that all three RNAi mutants missegregated chromosomes during the second meiotic division, though the effect was more pronounced in dcr1⁻ and rdp1⁻ strains. The results also revealed a small but consistent proportion of mutant cells that missegregated single chromatids during the first meiotic division.

TABLE 7
Frequency of Phenotypic Classes based on observations of cen-GFP
immunofluorescence during meiotic segregation.

Percentage of each Class

| Class | WT | ago1⁻ | dcr1⁻ | rdp1⁻ |
|---|---|---|---|---|
| I | 100 | 89.3 | 42.0 | 39.6 |
| II | 0 | 7.7 | 43.0 | 48.1 |
| III | 0 | 0.97 | 7.5 | 6.6 |
| IV | 0 | 1.9 | 7.6 | 5.7 |

Consistent with these observations, examination of tetrads after DAPI staining revealed frequent aberrations in the distribution of DNA to daughter spores. The most common phenotype observed was the presence of DNA outside of mature spores, indicating that one or more chromosomes failed to reach the meiotic pole and were not incorporated into the developing spore. These results implicate the RNAi machinery in the fidelity of meiotic chromosome segregation, an extremely important process that ensures the genomic integrity of future generations, and lies at the heart of many heritable human disorders. One possible explanation for the observed meiotic segregation defects is that, as during mitosis, loss of heterochromatin compromises centromere cohesion.

During the meiotic prophase of fission yeast, all of the telomeres cluster together near the spindle pole body and the nucleus becomes elongated and oscillates between the cells poles, led by the spindle pole body. This is referred to as the “horsetail” stage, and corresponds to the meiotic telomere bouquet observed in a wide range of eukaryotic systems (Zickler and Kleckner. Annu Rev Genet 1998; 32:619). Mutants affecting telomeric silencing have been shown, to varying degrees, to impair the meiotic clustering of telomeres near the spindle pole body, and generally to alter the progression of the horsetail stage (Nimmo et al. Nature 1998;392:825; Cooper et al. Nature 1998;392:828; Yamamoto and Hiraoka. BioEssays 2001;23:526; Chikashige and Hiraoka. Curr Biol 2001; 11:1618).

The role of RNAi machinery in telomere clustering in meiotic cells was examined by immunofluorescence with antibodies for Swi6 and Taz1 on meiotic cells in the horsetail stage, as identified by DAPI staining. Although the majority of mutant cells exhibited wild-type morphology, three classes of aberrant phenotypes were noted. Wild-type cells predominantly displayed morphology corresponding to class I, in which Taz1 and Swi6 colocalize to a single spot in the horsetail nucleus. Mutant strains revealed an approximately 2-fold elevation in the frequency of class II morphology, in which two distinct Taz1 spots were visible either as a doublet or as completely separate foci. Finally, class III consisted of a small but significant proportion of mutant cells in which greater than two telomere spots were visible (class III) indicating a loss of meiotic telomere organization. The frequency distribution of each class is shown in Table 8.

TABLE 8
Frequency of Phenotypic Classes based on observation of Taz1 and
Swi6 immunofluorescence during meiotic segregation.

Percentage of each Class

| Class | WT | ago1⁻ | dcr1⁻ | rdp1⁻ |
|---|---|---|---|---|
| I | 83.3 | 58.9 | 56.8 | 50.0 |
| II | 15.8 | 31.2 | 36.9 | 40.6 |
| III | 0.92 | 9.9 | 6.3 | 9.4 |

Mutants affecting meiotic telomere clustering frequently show aberrations in the localization of telomeres near the spindle pole body (SPB), the fungal equivalent to the centrosome (Jin et al. Genetics 2002;160:861). Immunofluorescence studies were carried out with Taz, as well as Sad1, an essential component of the SPB (Hagan and Yanagida. J Cell Biol 1995;1291033) to examine telomere attachment to the SPB. As expected, the single telomere cluster observed in wild-type cells and in mutant cells with class I morphology was always adjacent to the SPB. In cells containing two telomere clusters, each was associated with a distinct Sad1 spot, though frequently one of the two clusters was smaller than the other. In cells containing more than two telomere spots, no more than two of those telomere spots were associated with a visible Sad1 spot. These results indicate that mutations in the RNAi machinery lead to a mild but consistent disruption of meiotic telomere clustering and SPB integrity.

The observed defects in segregation and chromosome organization in RNAi mutants are most likely a product of changes in chromatin structure at centromeres and telomeres. This is consistent with the role disclosed herein for the RNAi machinery in the targeting of histone modifying activities.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description. Patents, patent applications, publications, product descriptions, and protocols are cited throughout this application, the disclosures of which are incorporated herein by reference in their entireties for all purposes.

1. A method of regulating the formation of heterochromatin at a target locus within the genome of a cell, which method comprises, introducing to the cell an RNAi probe that is complementary to a portion of the target locus, wherein said RNAi probe induces the assembly of heterochromatin at the region of the target locus that is complementary to the RNAi probe.

2. The method of claim 1, wherein the assembly of heterochromatin at the region of the target locus that is complementary to the RNAi probe produces heritable, pre-transcriptional silencing of a gene spanning the target locus.

3. The method of claim 1, wherein the assembly of heterochromatin at the region of the target locus that is complementary to the RNAi probe produces heritable, pre-transcriptional silencing of a gene adjacent to the target locus.

4. The method of claim 1, wherein the region of the target locus that is complementary to the RNAi probe is a promoter sequence.

5. A method of regulating the modification of a histone protein assembled on the DNA of a target locus within the genome of a cell, which method comprises, introducing to the cell an RNAi probe that is complementary to a portion of the target locus, wherein said RNAi probe induces the methylation of the histone protein assembled on the DNA at the region of the target locus that is complementary to the RNAi probe.

6. The method of claim 5, wherein the methylation of the histone protein assembled on the DNA at the region of the target locus that is complementary to the RNAi probe produces heritable, pre-transcriptional silencing of a gene spanning the target locus.

7. The method of claim 5, wherein the methylation of the histone protein assembled on the DNA at the region of the target locus that is complementary to the RNAi probe produces heritable, pre-transcriptional silencing of a gene adjacent to the target locus.

8. The method of claim 5, wherein the region of the target locus that is complementary to the RNAi probe is a promoter sequence.

9. A method of regulating the modification the DNA of a target locus within the genome of a cell, which method comprises, introducing to the cell an RNAi probe that is complementary to a portion of the target locus, wherein said RNAi probe induces the methylation of the DNA of a target locus at the region of the target locus that is complementary to the RNAi probe.

10. The method of claim 9, wherein the methylation of the DNA of a target locus at the region of the target locus that is complementary to the RNAi probe produces heritable, pre-transcriptional silencing of a gene spanning the target locus.

11. The method of claim 9, wherein the methylation of the DNA of a target locus at the region of the target locus that is complementary to the RNAi probe produces heritable, pre-transcriptional silencing of a gene adjacent to the target locus.

12. The method of claim 9, wherein the region of the target locus that is complementary to the RNAi probe is a promoter sequence.


SEQUENCE LISTING (24 sequences)

SEQ ID NO: 1 - DNA, 20 bp, artificial sequence
  primer
gttcagctgggatggatgat

SEQ ID NO: 2 - DNA, 20 bp, artificial sequence
  primer
ccctaacttggaaaggcaca

SEQ ID NO: 3 - DNA, 24 bp, artificial sequence
  primer
gaaaacacatcgttgtcttcagag

SEQ ID NO: 4 - DNA, 21 bp, artificial sequence
  primer
cgtcttgtagctgcatgtgaa

SEQ ID NO: 5 - DNA, 20 bp, artificial sequence
  primer
gctttatgccaaaacatgca

SEQ ID NO: 6 - DNA, 20 bp, artificial sequence
  primer
tgcatgttttggcataaagc

SEQ ID NO: 7 - DNA, 21 bp, artificial sequence
  primer
aaccaacgacatcatgggtag

SEQ ID NO: 8 - DNA, 21 bp, artificial sequence
  primer
ctacccatgatgtcgttggtt

SEQ ID NO: 9 - DNA, 23 bp, artificial sequence
  primer
tttgtggcataacaagttctcaa

SEQ ID NO: 10 - DNA, 19 bp, artificial sequence
  primer
aaacgaccaatatgctgcg

SEQ ID NO: 11 - DNA, 21 bp, artificial sequence
  primer
ccgcggtcttcttccgtgcgc

SEQ ID NO: 12 - DNA, 22 bp, artificial sequence
  primer
gcaagaatggatccaccaatcc

SEQ ID NO: 13 - DNA, 25 bp, artificial sequence
  primer
gcaatgttttgccaaagcgaaattg

SEQ ID NO: 14 - DNA, 27 bp, artificial sequence
  primer
tccaagactgttgttgagtgctgtgga

SEQ ID NO: 15 - DNA, 21 bp, artificial sequence
  primer
ccgcggtcttcttccgtgcgc

SEQ ID NO: 16 - DNA, 22 bp, artificial sequence
  primer
gcaagaatggatccaccaatcc

SEQ ID NO: 17 - DNA, 25 bp, artificial sequence
  primer
gcaatgttttgccaaagcgaaattg

SEQ ID NO: 18 - DNA, 27 bp, artificial sequence
  primer
tccaagactgttgttgagtgctgtgga

SEQ ID NO: 19 - DNA, 25 bp, artificial sequence
  primer
gcaatgttttgccaaagcgaaattg

SEQ ID NO: 20 - DNA, 27 bp, artificial sequence
  primer
tccaagactgttgttgagtgctgtgga

SEQ ID NO: 21 - DNA, 21 bp, artificial sequence
  primer
ccgcggtcttcttccgtgcgc

SEQ ID NO: 22 - DNA, 22 bp, artificial sequence
  primer
gcaagaatggatccaccaatcc

SEQ ID NO: 23 - DNA, 21 bp, artificial sequence
  primer
ccgcggtcttcttccgtgcgc

SEQ ID NO: 24 - DNA, 22 bp, artificial sequence
  primer
gcaagaatggatccaccaatcc